(19) United States
(12) Patent Application Publication
(10) Pub. No.: US 2022/0204521 A1
(43) Pub. Date: Jun. 30, 2022

(54) ELECTROACTIVE COMPOUNDS

(71) Applicant: DUPONT ELECTRONICS, INC., Wilmington, DE (US)

(72) Inventors: Viacheslav V DIEV, WILMINGTON, DE (US); Denis Yurievich KONDAKOV, WILMINGTON, DE (US); Yunlong ZOU, WILMINGTON, DE (US)

(21) Appl. No.: 17/594,459

(22) PCT Filed: May 5, 2020
(86) PCT No.: PCT/US2020/031409

§ 371 Date: Oct. 18, 2021

Related U.S. Application Data
(60) Provisional application No. 62/845,936, filed on May 10, 2019.

(51) Int. Cl. C07D 493/04; H01L 51/00; C07D 495/04; C07D 493/14; C07D 497/04

(52) U.S. Cl. CPC C07D 493/04; H01L 51/0073; C07D 495/04; H01L 51/5016; H01L 51/0074; H01L 51/0058; C07D 497/04; C07D 493/14

(57) ABSTRACT

There is provided a compound having Formula I

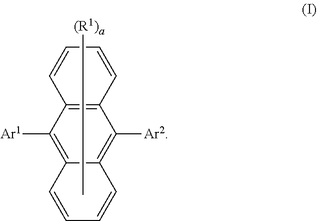

In Formula I: Ar¹ is a hydrocarbon aryl group, a heteroaryl group, or a substituted derivative thereof; and Are has Formula IA, IB, IC, IAa, IBb, or ICc

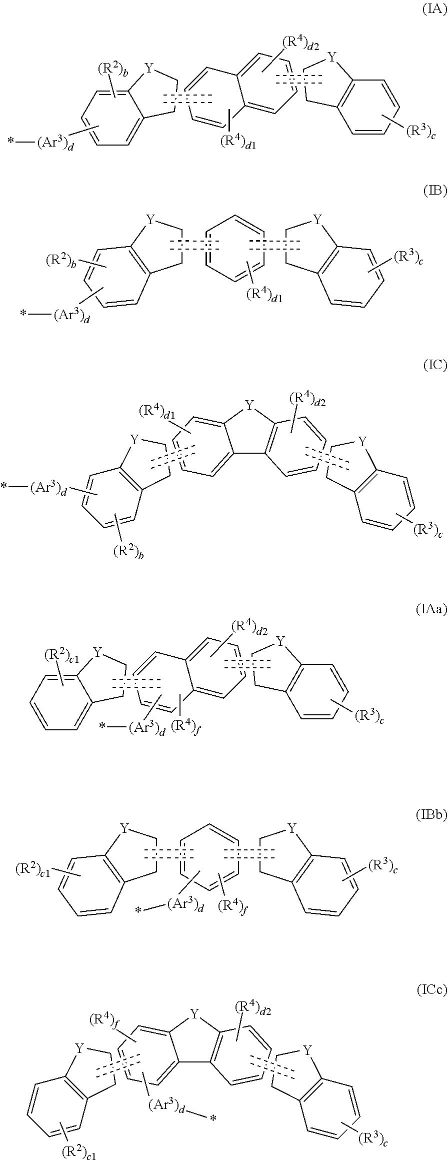

The variables are described in detail herein.

### CLAIM OF BENEFIT OF PRIOR APPLICATION

This application claims the benefit of U.S. Provisional Application No. 62/845,936, filed May 10, 2019, which is incorporated in its entirety herein by reference.

### BACKGROUND INFORMATION

### Field of the Disclosure

This disclosure relates in general to electroactive compounds and their use in electronic devices.

### Description of the Related Art

Organic electronic devices that emit light, such as light-emitting diodes that make up displays, are present in many different kinds of electronic equipment. In all such devices, an organic active layer is sandwiched between two electrical contact layers. At least one of the electrical contact layers is light-transmitting so that light can pass through the electrical contact layer. The organic active layer emits light through the light-transmitting electrical contact layer upon application of electricity across the electrical contact layers.

It is well known to use organic electroluminescent compounds as the active component in light-emitting diodes. Simple organic molecules, such as anthracene, thiadiazole derivatives, and coumarin derivatives are known to show electroluminescence. Metal complexes, particularly iridium and platinum complexes are also known to show electroluminescence. In some cases, these small molecule compounds are present as a dopant in a host material to improve processing and/or electronic properties.

There is a continuing need for new electroactive compounds that can be used as hosts or electroluminescent materials.

### SUMMARY

There is provided a compound having Formula I

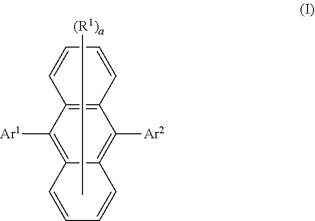

wherein:
Ar¹ is selected from the group consisting of hydrocarbon aryl groups, heteroaryl groups, and substituted derivatives thereof;
Ar² is selected from the group consisting of Formula IA, Formula IB, Formula IC, Formula IAa, Formula IBb, and Formula ICc

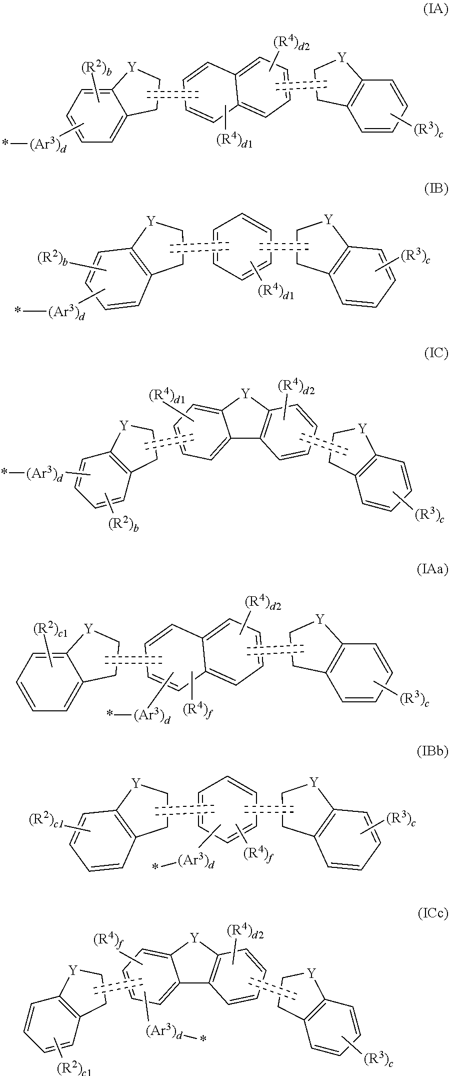

wherein:
Ar³ is the same or different at each occurrence and is selected from the group consisting of phenyl, naphthyl, and substituted derivatives thereof;
Y is the same or different at each occurrence and is selected from the group consisting of CR^{a}R^{b}, O, S, and Se, with the proviso that at least one Y is selected from the group consisting of O, S, and Se;
R^{a} and R^{b} are the same or different at each occurrence and are selected from the group consisting of alkyl, silyl, germyl, hydrocarbon aryl, heteroaryl, and substituted derivatives thereof, where R^{a} and R^{b} can be joined to form a cyclic group selected from the group consisting of cycloalkyl, silacycloalkyl, spirofluorenyl, silaspirofluorenyl, or a substituted derivative thereof;
R¹-R⁴ are the same or different at each occurrence and are selected from the group consisting of D, F, CN, alkyl, fluoroalkyl, hydrocarbon aryl, heteroaryl, silyl, germyl, deuterated alkyl, deuterated partially-fluorinated alkyl, deuterated hydrocarbon aryl, deuterated heteroaryl, deuterated heteroaryl deuterated silyl, and deuterated germyl, where adjacent R² and/or R³ groups can be joined together to form a fused aromatic ring;
a is an integer from 0-8;
b is an integer from 0-3;
c is an integer from 0-4;
d, d1, and d2 are the same or different and are an integer from 0-2;
f is an integer from 0-1;
a double dashed line between two rings indicates that the rings are fused together in any orientation; and
* indicates a point of attachment in the identified formula;
   with the proviso that:
in Formula IB and Formula IC there is at least one naphthyl group formed by R² or R³ groups, where the naphthyl group may have one or more substituents selected from the group consisting of D, F, CN, alkyl, fluoroalkyl, hydrocarbon aryl, heteroaryl, silyl, germyl, deuterated alkyl, deuterated partially-fluorinated alkyl, deuterated hydrocarbon aryl, deuterated heteroaryl, deuterated heteroaryl deuterated silyl, and deuterated germyl.

There is also provided an organic electronic device comprising a first electrical contact, a second electrical contact and a photoactive layer therebetween, the photoactive layer comprising a compound having Formula I.

The foregoing general description and the following detailed description are exemplary and explanatory only and are not restrictive of the invention, as defined in the appended claims.

### BRIEF DESCRIPTION OF THE DRAWINGS

Embodiments are illustrated in the accompanying figures to improve understanding of concepts as presented herein.

FIG. 1 includes an illustration of one example of an organic electronic device including a new compound described herein.

FIG. 2 includes an illustration of another example of an organic electronic device including a new compound described herein.

Skilled artisans appreciate that objects in the figures are illustrated for simplicity and clarity and have not necessarily been drawn to scale. For example, the dimensions of some of the objects in the figures may be exaggerated relative to other objects to help to improve understanding of embodiments.

### DETAILED DESCRIPTION

Many aspects and embodiments have been described above and are merely exemplary and not limiting. After reading this specification, skilled artisans appreciate that other aspects and embodiments are possible without departing from the scope of the invention.

Other features and benefits of any one or more of the embodiments will be apparent from the following detailed description, and from the claims. The detailed description first addresses Definitions and Clarification of Terms followed by the Compound Having Formula I, Devices, and finally Examples.

### 1. Definitions and Clarification of Terms

Before addressing details of embodiments described below, some terms are defined or clarified.

Unless otherwise specifically defined, R, R′, R″ and any other variables are generic designations. The specific definitions for a given formula herein are controlling for that formula.

The term “adjacent” as it refers to substituent groups refers to groups that are bonded to carbons that are joined together with a single or multiple bond. Exemplary adjacent R groups are shown below:

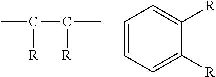

The term “alkoxy” is intended to mean the group RO—, where R is an alkyl group.

The term “alkyl” is intended to mean a group derived from an aliphatic hydrocarbon and includes a linear, a branched, or a cyclic group. A group “derived from” a compound, indicates the radical formed by removal of one or more H or D.

In some embodiments, an alkyl has from 1-20 carbon atoms.

The term “aromatic compound” is intended to mean an organic compound comprising at least one unsaturated cyclic group having 4n+2 delocalized pi electrons.

The term “aryl” is intended to mean a group derived from an aromatic hydrocarbon having one or more points of attachment. The term includes groups which have a single ring and those which have multiple rings which can be joined by a single bond or fused together. Hydrocarbon aryl groups have only carbon in the ring structures. Heteroaryl groups have at least one heteroatom in a ring structure.

The term “alkylaryl” is intended to mean an aryl group having one or more alkyl substituents.

The term “aryloxy” is intended to mean the group RO—, where R is an aryl group.

The term “charge transport,” when referring to a layer, material, member, or structure is intended to mean such layer, material, member, or structure facilitates migration of such charge through the thickness of such layer, material, member, or structure with relative efficiency and small loss of charge. Hole transport materials facilitate positive charge; electron transport materials facilitate negative charge. Although light-emitting materials may also have some charge transport properties, the term “charge transport layer, material, member, or structure” is not intended to include a layer, material, member, or structure whose primary function is light emission.

The term “deuterated” is intended to mean that at least one hydrogen (“H”) has been replaced by deuterium (“D”). The term “deuterated analog” refers to an analog of a compound or group having the same structure, but in which one or more available hydrogens have been replaced with deuterium. In a deuterated compound or deuterated analog, the deuterium is present in at least 100 times the natural abundance level. The term “% deuterated” or “% deuteration” is intended to mean the ratio of deuterons to the sum of protons plus deuterons, expressed as a percentage. The notation shown below

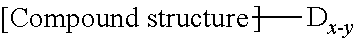

indicates that the compound is deuterated at any available position and that the total number of deuterium substituents is from x to y. For example, the compound shown below has 8-10 deuterium substituents at any available position

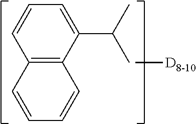

The term “dopant” is intended to mean a material, within a layer including a host material, that changes the electronic characteristic(s) or the targeted wavelength(s) of radiation emission, reception, or filtering of the layer compared to the electronic characteristic(s) or the wavelength(s) of radiation emission, reception, or filtering of the layer in the absence of such material.

The term “germyl” refers to the group R₃Ge—, where R is the same or different at each occurrence and is H, D, C1-20 alkyl, deuterated alkyl, fluoroalkyl, aryl, or deuterated aryl.

The prefix “hetero” indicates that one or more carbon atoms have been replaced with a different atom. In some embodiments, the different atom is N, O, or S.

The term “host material” is intended to mean a material, usually in the form of a layer, to which a dopant may be added. The host material may or may not have electronic characteristic(s) or the ability to emit, receive, or filter radiation.

The terms “luminescent material”, “emissive material” and “emitter” are intended to mean a material that emits light when activated by an applied voltage (such as in a light-emitting diode or light-emitting electrochemical cell). The term “blue luminescent material” is intended to mean a material capable of emitting radiation that has an emission maximum at a wavelength in a range of approximately 445-490 nm.

The term “layer” is used interchangeably with the term “film” and refers to a coating covering a desired area. The term is not limited by size. The area can be as large as an entire device or as small as a specific functional area such as the actual visual display, or as small as a single sub-pixel. Layers and films can be formed by any conventional deposition technique, including vapor deposition, liquid deposition (continuous and discontinuous techniques), and thermal transfer. Continuous deposition techniques, include but are not limited to, spin coating, gravure coating, curtain coating, dip coating, slot-die coating, spray coating, and continuous nozzle coating or printing. Discontinuous deposition techniques include, but are not limited to, ink jet printing, gravure printing, and screen printing.

The term “N-heterocycle” or “N-heteroaryl” refers to a heteroaromatic compound or group having at least one nitrogen in an aromatic ring.

The term “N,O,S-heterocycle” or “N,O,S-heteroaryl” refers to a heteroaromatic compound or group having at least one heteroatom in an aromatic ring, where the heteroatom is N, O, or S. The N,O,S-heterocycle may have more than one type of heteroatom.

The term “organic electronic device” or sometimes just “electronic device” is intended to mean a device including one or more organic semiconductor layers or materials.

The term “photoactive” refers to a material or layer that emits light when activated by an applied voltage (such as in a light emitting diode or chemical cell) or responds to radiant energy and generates a signal with or without an applied bias voltage (such as in a photodetector or a photovoltaic cell). The photoactive material or layer is sometimes referred to as the emissive layer. The photoactive layer is abbreviated herein as “EML”.

The term “silacycloalkyl” refers to a cyclic alkyl group where one or more carbons have been replaced with silicons.

The term “silaspirofluorenyl” refers to a spirofluorenyl group where the spiro carbon has been replaced with silicon.

The term “siloxane” refers to the group R₃SiO(R₂Si)—, where R is the same or different at each occurrence and is H, D, C1-20 alkyl, deuterated alkyl, fluoroalkyl, aryl, or deuterated aryl. In some embodiments, one or more carbons in an R alkyl group are replaced with Si.

The term “siloxy” refers to the group R₃SiO—, where R is the same or different at each occurrence and is H, D, C1-20 alkyl, deuterated alkyl, fluoroalkyl, aryl, or deuterated aryl.

The term “silyl” refers to the group R₃Si—, where R is the same or different at each occurrence and is H, D, C1-20 alkyl, deuterated alkyl, fluoroalkyl, aryl, or deuterated aryl. In some embodiments, one or more carbons in an R alkyl group are replaced with Si.

The term “spirofluorenyl” refers to a group derived from the compound below, where the central carbon is referred to as the spiro carbon.

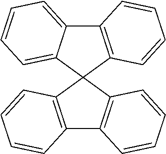 [SMILES: c1ccc2c(c1)-c1ccccc1C21c2ccccc2-c2ccccc21]

All groups may be unsubstituted or substituted. The substituent groups are discussed below. In a structure where a substituent bond passes through one or more rings as shown below,

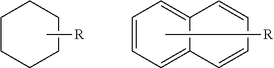

it is meant that the substituent R may be bonded at any available position on the one or more rings.

In any of the formulas or combination of formulas below, any subscript, such as a-h, k, p, q, r, s, a1, b1, and k1, that is present more than one time, may be the same or different at each occurrence.

In this specification, unless explicitly stated otherwise or indicated to the contrary by the context of usage, where an embodiment of the subject matter hereof is stated or described as comprising, including, containing, having, being composed of or being constituted by or of certain features or elements, one or more features or elements in addition to those explicitly stated or described may be present in the embodiment. An alternative embodiment of the disclosed subject matter hereof, is described as consisting essentially of certain features or elements, in which embodiment features or elements that would materially alter the principle of operation or the distinguishing characteristics of the embodiment are not present therein. A further alternative embodiment of the described subject matter hereof is described as consisting of certain features or elements, in which embodiment, or in insubstantial variations thereof, only the features or elements specifically stated or described are present.

Also, use of “a” or “an” are employed to describe elements and components described herein. This is done merely for convenience and to give a general sense of the scope of the invention. This description should be read to include one or at least one and the singular also includes the plural unless it is obvious that it is meant otherwise.

Group numbers corresponding to columns within the Periodic Table of the elements use the “New Notation” convention as seen in the CRC Handbook of Chemistry and Physics, 81^{st} Edition (2000-2001).

Unless otherwise defined, all technical and scientific terms used herein have the same meaning as commonly understood by one of ordinary skill in the art to which this invention belongs. Although methods and materials similar or equivalent to those described herein can be used in the practice or testing of embodiments of the present invention, suitable methods and materials are described below. In addition, the materials, methods, and examples are illustrative only and not intended to be limiting.

To the extent not described herein, many details regarding specific materials, processing acts, and circuits are conventional and may be found in textbooks and other sources within the organic light-emitting diode display, photodetector, photovoltaic cell, and semiconductive member arts.

### 2. Compounds Having Formula I

In some embodiments, the compounds described herein have Formula I

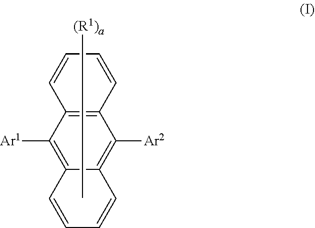

wherein:
Ar¹ is selected from the group consisting of hydrocarbon aryl groups, heteroaryl groups, and substituted derivatives thereof;
Ar² is selected from the group consisting of Formula IA, Formula IB,

Formula IC, Formula IAa, Formula IBb, and Formula ICc

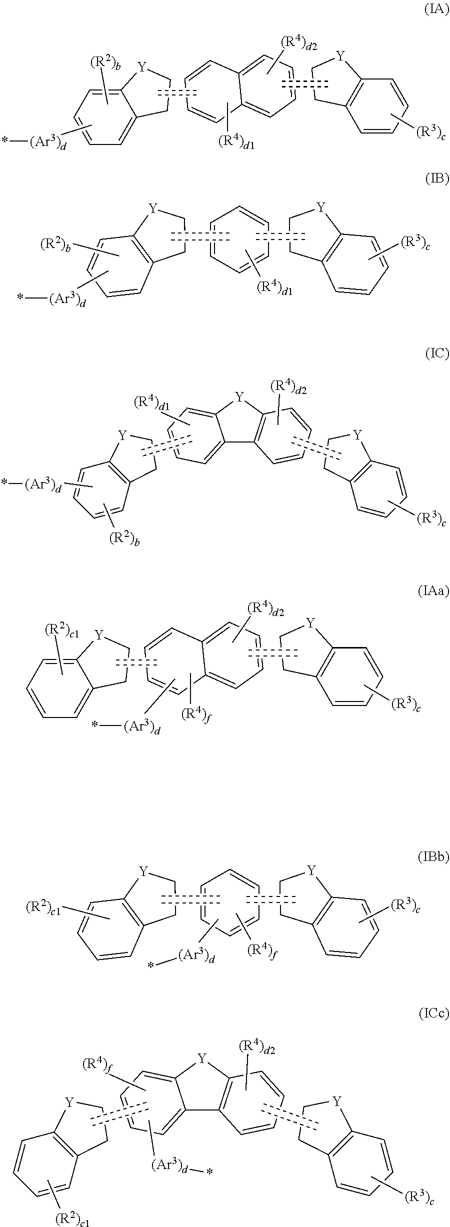

wherein:
Ar³ is the same or different at each occurrence and is selected from the group consisting of phenyl, naphthyl, and substituted derivatives thereof;
Y is the same or different at each occurrence and is selected from the group consisting of CR^{a}R^{b}, O, S, and Se, with the proviso that at least one Y is selected from the group consisting of O, S, and Se;
R^{a} and R^{b} are the same or different at each occurrence and are selected from the group consisting of alkyl, silyl, germyl, hydrocarbon aryl, heteroaryl, and substituted derivatives thereof, where R^{a} and R^{b} can be joined to form a cyclic group selected from the group consisting of cycloalkyl, silacycloalkyl, spirofluorenyl, silaspirofluorenyl, or a substituted derivative thereof;
R¹-R⁴ are the same or different at each occurrence and are selected from the group consisting of D, F, CN, alkyl, fluoroalkyl, hydrocarbon aryl, heteroaryl, silyl, germyl, deuterated alkyl, deuterated partially-fluorinated alkyl, deuterated hydrocarbon aryl, deuterated heteroaryl, deuterated heteroaryl deuterated silyl, and deuterated germyl, where adjacent R² and/or R³ groups can be joined together to form a fused aromatic ring;
a is an integer from 0-8;
b is an integer from 0-3;
c is an integer from 0-4;
d, d1, and d2 are the same or different and are an integer from 0-2;
f is an integer from 0-1;
a double dashed line between two rings indicates that the rings are fused together in any orientation; and
* indicates a point of attachment in the identified formula;
   with the proviso that:
in Formula IB and Formula IC there is at least one naphthyl group formed by R² or R³ groups, where the naphthyl group may have one or more substituents selected from the group consisting of D, F, CN, alkyl, fluoroalkyl, hydrocarbon aryl, heteroaryl, silyl, germyl, deuterated alkyl, deuterated partially-fluorinated alkyl, deuterated hydrocarbon aryl, deuterated heteroaryl, deuterated heteroaryl deuterated silyl, and deuterated germyl.

In some embodiments, the compounds having Formula I are readily sublimable. This is advantageous for purification and for vapor deposition.

In some embodiments, devices including the compounds of Formula I have low operating voltage. In some embodiments, the voltage is less than 5 V at 10 mA/cm²; in some embodiments, less than 4 V at 10 mA/cm².

In some embodiments of Formula I, the compound is deuterated. In some embodiments, the compound is at least 10% deuterated; in some embodiments, at least 20% deuterated; in some embodiments, at least 30% deuterated; in some embodiments, at least 40% deuterated; in some embodiments, at least 50% deuterated; in some embodiments, at least 60% deuterated; in some embodiments, at least 70% deuterated; in some embodiments, at least 80% deuterated; in some embodiments, at least 90% deuterated; in some embodiments, 100% deuterated.

In some embodiments of Formula I, deuteration is present on the anthracene core group.

In some embodiments of Formula I, deuteration is present on one or both of Ar¹ and Ar².

In some embodiments of Formula I, Ar¹ is selected from the group consisting of hydrocarbon aryl groups, heteroaryl groups, and substituted derivatives thereof, wherein substituted derivatives have only substituents selected from the group consisting of D, alkyl, silyl, germyl, deuterated alkyl, deuterated silyl, and deuterated germyl, and no other substituents.

In some embodiments of Formula I, Ar¹ is an unsubstituted hydrocarbon aryl.

In some embodiments of Formula I, Ar¹ is a hydrocarbon aryl or deuterated analog thereof having 6-30 ring carbons; in some embodiments 6-18 ring carbons.

In some embodiments of Formula I, Ar¹ is a substituted hydrocarbon aryl, where the substituent is selected from the group consisting of D, alkyl, silyl, germyl, hydrocarbon aryl, heteroaryl, deuterated alkyl, deuterated silyl, deuterated germyl, deuterated hydrocarbon aryl, and deuterated heteroaryl. In some embodiments, the heteroaryl has heteroatoms selected from the group consisting of O, S, and Se.

In some embodiments of Formula I, Ar¹ is selected from the group consisting of phenyl, biphenyl, terphenyl, 1-naphthyl, 2-naphthyl, anthracenyl, fluorenyl, phenanthryl, deuterated analogs thereof, and derivatives thereof having one or more substituents selected from the group consisting of D, alkyl, silyl, germyl, hydrocarbon aryl, heteroaryl, deuterated alkyl, deuterated silyl, deuterated germyl, deuterated hydrocarbon aryl, and deuterated heteroaryl. In some embodiments, the heteroaryl has heteroatoms selected from the group consisting of O, S, and Se.

In some embodiments of Formula I, Ar¹ is selected from the group consisting of phenyl, biphenyl, terphenyl, 1-naphthyl, 2-naphthyl, anthracenyl, fluorenyl, phenanthryl, and derivatives thereof having one or more substituents selected from the group consisting of D, alkyl, silyl, germyl, deuterated alkyl, deuterated silyl, and deuterated germyl.

In some embodiments of Formula I, Ar¹ is selected from the group consisting of phenyl, biphenyl, naphthyl and substituted derivatives thereof. In some embodiments of Formula I, Ar¹ is selected from the group consisting of phenyl, biphenyl, naphthyl and deuterated analogs thereof.

In some embodiments of Formula I, Ar¹ is an unsubstituted heteroaryl.

In some embodiments of Formula I, Ar¹ is a heteroaryl or deuterated analog thereof having 3-30 ring carbons; in some embodiments 3-18 ring carbons.

In some embodiments of Formula I, Ar¹ is a substituted heteroaryl, where the substituent is selected from the group consisting of D, alkyl, silyl, germyl, deuterated alkyl, deuterated silyl, and deuterated germyl.

In some embodiments of Formula I, Ar¹ is selected from the group consisting of heteroaryl and deuterated heteroaryl, where the heteroaryl has at least one ring atom which is selected from the group consisting of O and S.

In some embodiments of Formula I, Ar¹ is an O-heteroaryl having at least one ring atom that is 0.

In some embodiments, the O-heteroaryl is derived from a compound selected from the group consisting of furan, benzofuran, isobenzofuran, dibenzofuran, and substituted derivatives thereof.

In some embodiments of Formula I, Ar¹ is present and is an S-heteroaryl having at least one ring atom which is S.

In some embodiments, the S-heteroaryl is derived from a compound selected form the group consisting of thiophene, benzothiophene, isobenzothiophene, dibenzothiophene, and substituted derivatives thereof.

In some embodiments of Formula I, Ar¹ has a formula selected from Formula IA through Formula IC, described in detail below.

In some embodiments of Formula I, Ar¹=Ar².

In some embodiments of Formula I, Ar¹≠Ar².

In some embodiments of Formula I, a=0.

In some embodiments of Formula I, a=1.

In some embodiments of Formula I, a=2.

In some embodiments of Formula I, a=3.

In some embodiments of Formula I, a=4.

In some embodiments of Formula I, a=5.

In some embodiments of Formula I, a=6.

In some embodiments of Formula I, a=7.

In some embodiments of Formula I, a=8.

In some embodiments of Formula I, a>0.

In some embodiments of Formula I, a>0 and at least one R¹ is selected from the group consisting of D, alkyl, silyl, deuterated alkyl, and deuterated silyl.

In some embodiments of Formula I, a>0 and at least one R¹=D.

In some embodiments of Formula I, a>0 and at least one R¹ is a C₁₋₁₀ alkyl or deuterated alkyl.

In some embodiments of Formula I, a>0 and at least one R¹ is a C₁₋₁₀ silyl or deuterated silyl.

In some embodiments of Formula I, Ar² has Formula IA

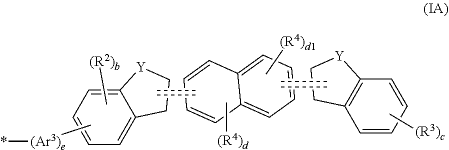

as defined above.

In some embodiments of Formula IA, both Y's are heteroatoms selected from the group consisting of O, S, and Se.

In some embodiments of Formula IA, both Y's are the same and are a heteroatom.

In some embodiments of Formula IA, the Y's are different.

In some embodiments of Formula IA, one Y=CR^{a}R^{b}.

In some embodiments of Formula IA, R^{a} is a substituted or unsubstituted alkyl having 1-20 carbon atoms or deuterated analog thereof; in some embodiments, 1-10 carbons. In some embodiments, the substituted alkyl has one or more substituents selected from the group consisting of D, hydrocarbon aryl, and deuterated hydrocarbon aryl.

In some embodiments of Formula IA, R^{a} is an unsubstituted or substituted hydrocarbon aryl having 6-30 ring carbons; in some embodiments, 6-12 ring carbons. In some embodiments, the substituted hydrocarbon aryl has one or more substituents selected from the group consisting of D, alkyl, silyl, germyl, deuterated alkyl, deuterated silyl, and deuterated germyl.

In some embodiments of Formula IA, R^{a} is an unsubstituted or substituted silyl group having 3-10 carbons. In some embodiments, the substituent is selected from the group consisting of D, hydrocarbon aryl, and deuterated hydrocarbon aryl.

All of the above-described embodiments for R^{a} apply equally to R^{b}.

In some embodiments of Formula IA, R^{a} and R^{b} are joined to form a cyclic group selected from the group consisting of cycloalkyl, spirofluorenyl, and a substituted derivative thereof, wherein the substituents are selected from the group consisting of D, alkyl, silyl, germyl, deuterated alkyl, deuterated silyl, and deuterated germyl.

In some embodiments of Formula IA, at least one Y=O.

In some embodiments of Formula IA, at least one Y=S.

In some embodiments of Formula IA, at least one Y=Se.

In some embodiments of Formula IA, both Y=O.

In some embodiments of Formula IA, both Y=S.

In some embodiments of Formula IA, both Y=Se.

In some embodiments of Formula IA, d=0.

In some embodiments of Formula IA, d=1.

In some embodiments of Formula IA, d=2.

In some embodiments of Formula IA, d>0.

In some embodiments of Formula IA, d>0 and Ar³ is an unsubstituted phenyl group. As used herein, the term “phenyl” includes groups having one or more points of attachment.

In some embodiments of Formula IA, d>0 and at least one Ar³ is a substituted phenyl group, where the substituent is selected from the group consisting of D, alkyl, silyl, germyl, deuterated alkyl, deuterated silyl, and deuterated germyl.

In some embodiments of Formula IA, d>0 and at least one Ar³ is an unsubstituted naphthyl group. As used herein, the term “naphthyl” includes groups having one or more points of attachment.

In some embodiments of Formula IA, d>0 and at least one Ar³ is a substituted naphthyl group, where the substituent is selected from the group consisting of D, alkyl, silyl, germyl, deuterated alkyl, deuterated silyl, and deuterated germyl.

In some embodiments of Formula IA, d=1 and Ar³ is selected from the group consisting of phenyl, biphenyl, 1-naphthyl, 2-naphthyl, and derivatives thereof having one or more substituents selected from the group consisting of D, alkyl, silyl, germyl, deuterated alkyl, deuterated silyl, and deuterated germyl.

In some embodiments of Formula IA, b=0.

In some embodiments of Formula IA, b=1.

In some embodiments of Formula IA, b=2.

In some embodiments of Formula IA, b=3.

In some embodiments of Formula IA, b>0.

In some embodiments of Formula IA, b>0 and at least one R² is D.

In some embodiments of Formula IA, b>0 and at least one R² is a hydrocarbon aryl or substituted derivative having 6-18 ring carbons.

In some embodiments of Formula IA, b>0 and at least one R² is selected from the group consisting of phenyl, biphenyl, terphenyl, alkyl-substituted derivatives thereof, silyl-substituted derivatives thereof, and deuterated analogs thereof.

In some embodiments of Formula IA, b>0 and at least one R² is selected from the group consisting of phenyl, biphenyl, terphenyl, alkyl-substituted derivatives thereof, silyl-substituted derivatives thereof, and deuterated analogs thereof.

In some embodiments of Formula IA, d1=0.

In some embodiments of Formula IA, d1=1.

In some embodiments of Formula IA, d1=2.

In some embodiments of Formula IA, d1>0.

In some embodiments of Formula IA, d2=0.

In some embodiments of Formula IA, d2=1.

In some embodiments of Formula IA, d2=2.

In some embodiments of Formula IA, d2>0.

In some embodiments of Formula IA, at least one of d1 and d2 is greater than 0 and at least one R⁴ is D.

In some embodiments of Formula IA, at least one of d1 and d2 is greater than 0 and at least one R⁴ is a hydrocarbon aryl or substituted derivative having 6-18 ring carbons.

In some embodiments of Formula IA, at least one of d1 and d2 is greater than 0 and at least one R⁴ is selected from the group consisting of phenyl, biphenyl, terphenyl, alkyl-substituted derivatives thereof, silyl-substituted derivatives thereof, and deuterated analogs thereof.

In some embodiments of Formula IA, at least one of d1 and d2 is greater than 0 and at least one R⁴ is selected from the group consisting of phenyl, biphenyl, terphenyl, alkyl-substituted derivatives thereof, silyl-substituted derivatives thereof, and deuterated analogs thereof.

In some embodiments of Formula IA, d1=d2=0.

In some embodiments of Formula IA, c=0.

In some embodiments of Formula IA, c=1.

In some embodiments of Formula IA, c=2.

In some embodiments of Formula IA, c=3.

In some embodiments of Formula IA, c=4.

In some embodiments of Formula IA, c>0.

In some embodiments of Formula IA, c>0 and at least one R³ is D.

In some embodiments of Formula IA, c>0 and at least one R³ is a hydrocarbon aryl or substituted derivative having 6-18 ring carbons.

In some embodiments of Formula IA, c>0 and at least one R³ is selected from the group consisting of phenyl, biphenyl, terphenyl, alkyl-substituted derivatives thereof, silyl-substituted derivatives thereof, and deuterated analogs thereof.

In some embodiments of Formula IA, c>0 and at least one R³ is selected from the group consisting of phenyl, biphenyl, terphenyl, alkyl-substituted derivatives thereof, silyl-substituted derivatives thereof, and deuterated analogs thereof.

In some embodiments of Formula IA, b≥2 and two adjacent R² groups are joined together to form a fused aromatic ring, thus forming a naphthyl group. The naphthyl group can be further substituted with one or more substituents selected from the group consisting of D, F, CN, alkyl, fluoroalkyl, hydrocarbon aryl, heteroaryl, silyl, germyl, deuterated alkyl, deuterated partially-fluorinated alkyl, deuterated hydrocarbon aryl, deuterated heteroaryl, deuterated heteroaryl deuterated silyl, and deuterated germyl. The ring can be fused at any available position, as shown below.

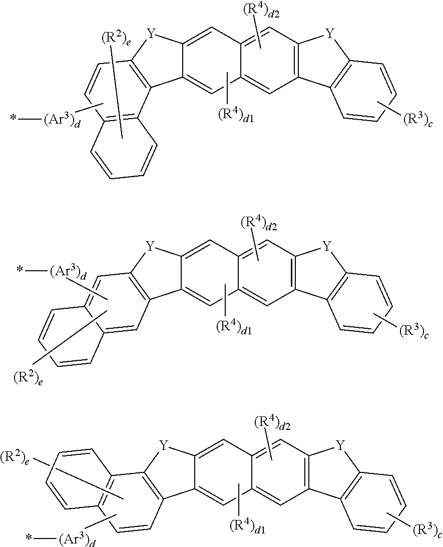

In the above formulas e is an integer from 0-5 and Y, Ar³, R², R³, R⁴, c, d1, and d2 are as defined in Formula IA.

In some embodiments of Formula IA, c≥2 and two adjacent R³ groups are joined together to form a fused aromatic ring, thus forming a naphthyl group. The naphthyl group can be further substituted with one or more substituents selected from the group consisting of D, F, CN, alkyl, fluoroalkyl, hydrocarbon aryl, heteroaryl, silyl, germyl, deuterated alkyl, deuterated partially-fluorinated alkyl, deuterated hydrocarbon aryl, deuterated heteroaryl, deuterated heteroaryl deuterated silyl, and deuterated germyl. The ring can be fused at any available position.

In some embodiments of Formula IA, b≥2, two adjacent R² groups are joined together to form a fused aromatic ring, c≥2, and two adjacent R³ groups are joined together to form a fused aromatic ring, thus forming two naphthyl groups. The naphthyl groups can be further substituted with one or more substituents selected from the group consisting of D, F, CN, alkyl, fluoroalkyl, hydrocarbon aryl, heteroaryl, silyl, germyl, deuterated alkyl, deuterated partially-fluorinated alkyl, deuterated hydrocarbon aryl, deuterated heteroaryl, deuterated heteroaryl deuterated silyl, and deuterated germyl. The rings can be fused at any available position.

In some embodiments, Formula IA has Formula IA-1, Formula IA-2, Formula IA-3, or Formula IA-4

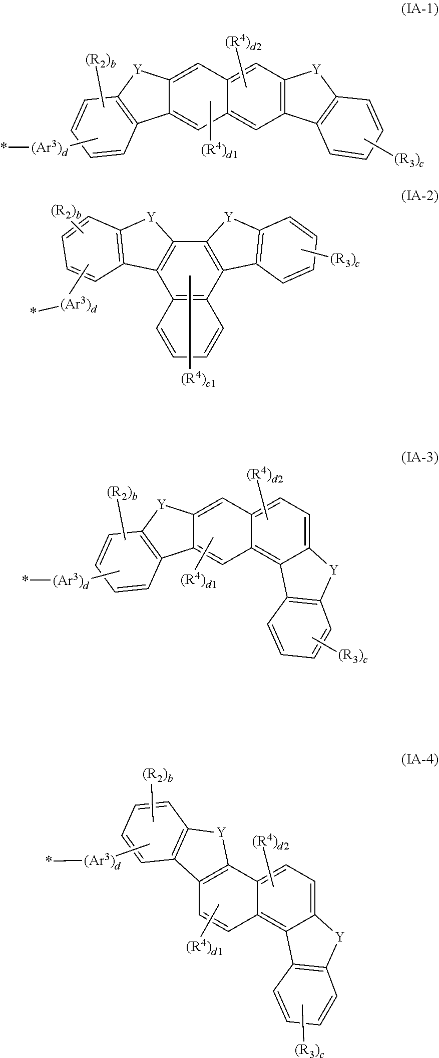

where:
Ar³ is the same or different at each occurrence and is selected from the group consisting of phenyl, naphthyl, and substituted derivatives thereof;
Y is the same or different at each occurrence and is selected from the group consisting of CR^{a}R^{b}, O, S, and Se, with the proviso that at least one Y is selected from the group consisting of O, S, and Se;
R^{a} and R^{b} are the same or different at each occurrence and are selected from the group consisting of alkyl, silyl, germyl, hydrocarbon aryl, heteroaryl, and substituted derivatives thereof, where R^{a} and R^{b} can be joined to form a cyclic group selected from the group consisting of cycloalkyl, silacycloalkyl, spirofluorenyl, silaspirofluorenyl, or a substituted derivative thereof;
R²-R⁴ are the same or different at each occurrence and are selected from the group consisting of D, F, CN, alkyl, fluoroalkyl, hydrocarbon aryl, heteroaryl, silyl, germyl, deuterated alkyl, deuterated partially-fluorinated alkyl, deuterated hydrocarbon aryl, deuterated heteroaryl, deuterated heteroaryl deuterated silyl, and deuterated germyl, where adjacent R² and/or R³ groups can be joined together to form a fused aromatic ring;
b is an integer from 0-3;
c and c1 are the same or different and are an integer from 0-4;
d, d1, and d2 are the same or different and are an integer from 0-2; and
* indicates a point of attachment in the identified formula.

In some embodiments of Formula IA-2, c1=0.

In some embodiments of Formula IA-2, c1=1.

In some embodiments of Formula IA-2, c1=2.

In some embodiments of Formula IA-2, c1=3.

In some embodiments of Formula IA-2, c1=4.

In some embodiments of Formula IA-2, c1>0.

All of the above-described embodiments for Y, Ar³, R², R³, R⁴, b, c, d, d1, and d2 in Formula IA, apply equally to Y, Ar³, R², R³, R⁴, b, c, d, d1, and d2 in Formulas IA-1 through IA-4.

In some embodiments of Formula I, Ar² has Formula IB

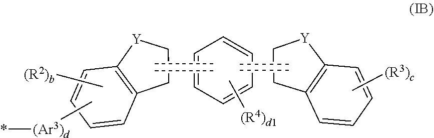

as defined above.

In Formula IB, there is at least one naphthyl group formed by R² or R³ substituents.

In some embodiments of Formula IB, b≥2 and two adjacent R² groups are joined together to form a fused aromatic ring, thus forming a naphthyl group. The naphthyl group can be further substituted with one or more substituents selected from the group consisting of D, F, CN, alkyl, fluoroalkyl, hydrocarbon aryl, heteroaryl, silyl, germyl, deuterated alkyl, deuterated partially-fluorinated alkyl, deuterated hydrocarbon aryl, deuterated heteroaryl, deuterated heteroaryl deuterated silyl, and deuterated germyl. The ring can be fused at any available position, as shown below.

In some embodiments of Formula IB, c≥2 and two adjacent R³ groups are joined together to form a fused aromatic ring, thus forming a naphthyl group. The naphthyl group can be further substituted with one or more substituents selected from the group consisting of D, F, CN, alkyl, fluoroalkyl, hydrocarbon aryl, heteroaryl, silyl, germyl, deuterated alkyl, deuterated partially-fluorinated alkyl, deuterated hydrocarbon aryl, deuterated heteroaryl, deuterated heteroaryl deuterated silyl, and deuterated germyl. The ring can be fused at any available position.

In some embodiments of Formula IB, b≥2, two adjacent R² groups are joined together to form a fused aromatic ring, c≥2, and two adjacent R³ groups are joined together to form a fused aromatic ring, thus forming two naphthyl groups. The naphthyl groups can be further substituted with one or more substituents selected from the group consisting of D, F, CN, alkyl, fluoroalkyl, hydrocarbon aryl, heteroaryl, silyl, germyl, deuterated alkyl, deuterated partially-fluorinated alkyl, deuterated hydrocarbon aryl, deuterated heteroaryl, deuterated heteroaryl deuterated silyl, and deuterated germyl. The rings can be fused at any available position.

All of the above-described embodiments for Y, Ar³, R², R³, R⁴, b, c, d, and d1 in Formula IA, apply equally to Y, Ar³, R², R³, R⁴, b, c, d, and d1 in Formula IB.

In some embodiments, Formula IB has Formula IB-1 Formula IB-2, or Formula IB-3

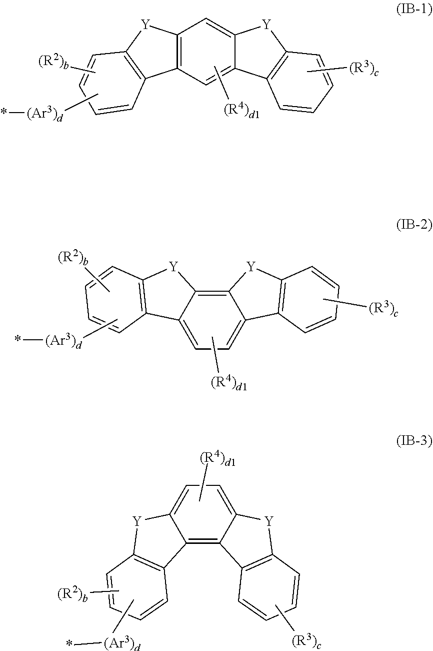

where:
Ar³ is the same or different at each occurrence and is selected from the group consisting of phenyl, naphthyl, and substituted derivatives thereof;
Y is the same or different at each occurrence and is selected from the group consisting of CR^{a}R^{b}, O, S, and Se, with the proviso that at least one Y is selected from the group consisting of O, S, and Se;
R^{a} and R^{b} are the same or different at each occurrence and are selected from the group consisting of alkyl, silyl, germyl, hydrocarbon aryl, heteroaryl, and substituted derivatives thereof, where R^{a} and R^{b} can be joined to form a cyclic group selected from the group consisting of cycloalkyl, silacycloalkyl, spirofluorenyl, silaspirofluorenyl, or a substituted derivative thereof;
R²-R⁴ are the same or different at each occurrence and are selected from the group consisting of D, F, CN, alkyl, fluoroalkyl, hydrocarbon aryl, heteroaryl, silyl, germyl, deuterated alkyl, deuterated partially-fluorinated alkyl, deuterated hydrocarbon aryl, deuterated heteroaryl, deuterated heteroaryl deuterated silyl, and deuterated germyl, where adjacent R² and/or R³ groups can be joined together to form a fused aromatic ring;
b is an integer from 0-3;
c and c1 are the same or different and are an integer from 0-4;
d and d1 are the same or different and are an integer from 0-2; and
* indicates a point of attachment in the identified formula;
with the proviso that there is at least one naphthyl group formed by R² or R³ groups, where the naphthyl group may have one or more substituents selected from the group consisting of D, F, CN, alkyl, fluoroalkyl, hydrocarbon aryl, heteroaryl, silyl, germyl, deuterated alkyl, deuterated partially-fluorinated alkyl, deuterated hydrocarbon aryl, deuterated heteroaryl, deuterated heteroaryl deuterated silyl, and deuterated germyl.

All of the above-described embodiments for Y, Ar³, R², R³, R⁴, b, c, d, and d1 in Formula IA, apply equally to Y, Ar³, R², R³, R⁴, b, c, d, and d1 in Formula IB-1, Formula IB-2, and Formula IB-3.

In some embodiments of Formula I, Ar² has Formula IC

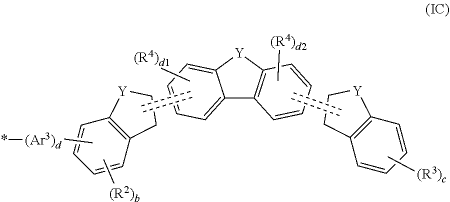

as defined above.

In Formula IC, there is at least one naphthyl group formed by R² or R³ substituents.

In some embodiments of Formula IA, b≥2 and two adjacent R² groups are joined together to form a fused aromatic ring, thus forming a naphthyl group. The naphthyl group can be further substituted with one or more substituents selected from the group consisting of D, F, CN, alkyl, fluoroalkyl, hydrocarbon aryl, heteroaryl, silyl, germyl, deuterated alkyl, deuterated partially-fluorinated alkyl, deuterated hydrocarbon aryl, deuterated heteroaryl, deuterated heteroaryl deuterated silyl, and deuterated germyl. The ring can be fused at any available position.

In some embodiments of Formula IC, c≥2 and two adjacent R³ groups are joined together to form a fused aromatic ring, thus forming a naphthyl group. The naphthyl group can be further substituted with one or more substituents selected from the group consisting of D, F, CN, alkyl, fluoroalkyl, hydrocarbon aryl, heteroaryl, silyl, germyl, deuterated alkyl, deuterated partially-fluorinated alkyl, deuterated hydrocarbon aryl, deuterated heteroaryl, deuterated heteroaryl deuterated silyl, and deuterated germyl. The ring can be fused at any available position.

In some embodiments of Formula IC, b≥2, two adjacent R² groups are joined together to form a fused aromatic ring, c 2, and two adjacent R³ groups are joined together to form a fused aromatic ring, thus forming two naphthyl groups. The naphthyl groups can be further substituted with one or more substituents selected from the group consisting of D, F, CN, alkyl, fluoroalkyl, hydrocarbon aryl, heteroaryl, silyl, germyl, deuterated alkyl, deuterated partially-fluorinated alkyl, deuterated hydrocarbon aryl, deuterated heteroaryl, deuterated heteroaryl deuterated silyl, and deuterated germyl. The rings can be fused at any available position.

In some embodiments of Formula IC, all three Y's are heteroatoms selected from the group consisting of O, S, and Se.

In some embodiments of Formula IC, all three Y's are different from each other.

In some embodiments of Formula IC, two Y's are the same.

In some embodiments of Formula IC, all three Y's are the same and are a heteroatom.

In some embodiments of Formula IC, at least one Y=CR^{a}R^{b}. All of the above-described embodiments for R^{a} and R^{b} in Formula IA apply equally to R^{a} and R^{b} in Formula IC.

In some embodiments of Formula IC, at least one Y=O,

In some embodiments of Formula IC, at least one Y=S.

In some embodiments of Formula IC, at least one Y=Se.

In some embodiments of Formula IC, all Y=O.

In some embodiments of Formula IC, all Y=S.

In some embodiments of Formula IC, all Y=Se.

All of the above-described embodiments for Ar³, R², R³, R⁴, b, c, d, d1, and d2 in Formula IA, apply equally to Y, Ar³, R², R³, R⁴, b, c, d, d1, and d2 in Formula IC.

In some embodiments, Formula IC has one of Formula IC-1 through Formula IC-9

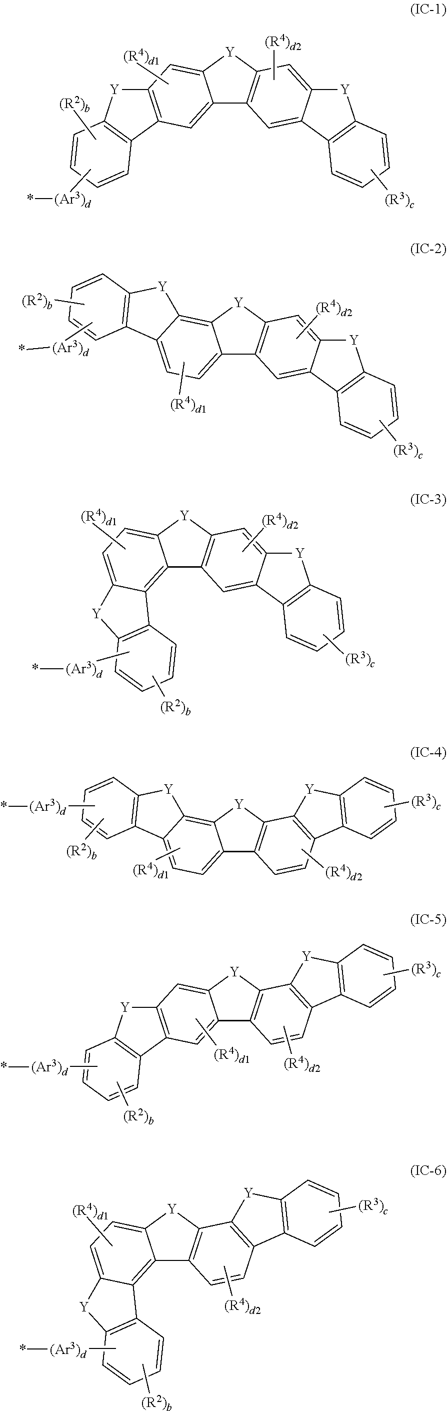

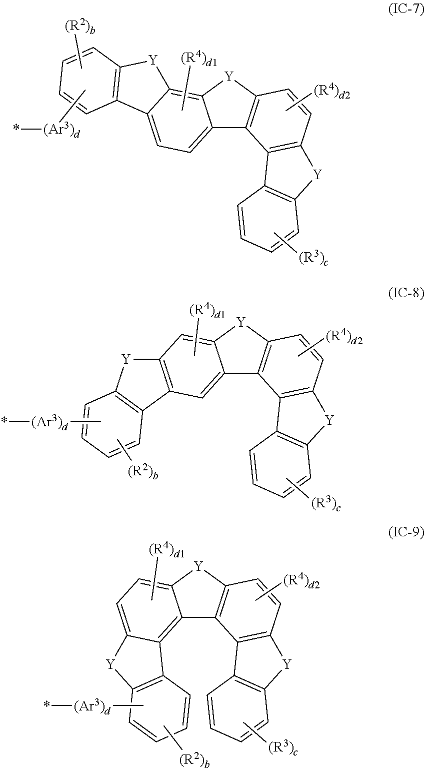

where:
Ar^{a} is the same or different at each occurrence and is selected from the group consisting of phenyl, naphthyl, and substituted derivatives thereof;
Y is the same or different at each occurrence and is selected from the group consisting of CR^{a}R^{b}, O, S, and Se, with the proviso that at least one Y is selected from the group consisting of O, S, and Se;
R^{a} and R^{b} are the same or different at each occurrence and are selected from the group consisting of alkyl, silyl, germyl, hydrocarbon aryl, heteroaryl, and substituted derivatives thereof, where R^{a} and R^{b} can be joined to form a cyclic group selected from the group consisting of cycloalkyl, silacycloalkyl, spirofluorenyl, silaspirofluorenyl, or a substituted derivative thereof;
R²-R⁴ are the same or different at each occurrence and are selected from the group consisting of D, F, CN, alkyl, fluoroalkyl, hydrocarbon aryl, heteroaryl, silyl, germyl, deuterated alkyl, deuterated partially-fluorinated alkyl, deuterated hydrocarbon aryl, deuterated heteroaryl, deuterated heteroaryl deuterated silyl, and deuterated germyl, where adjacent R² and/or R³ groups can be joined together to form a fused aromatic ring;
b is an integer from 0-3;
c and c1 are the same or different and are an integer from 0-4;
d, d1, and d2 are the same or different and are an integer from 0-2; and
* indicates a point of attachment in the identified formula;
with the proviso that there is at least one naphthyl group formed by R² or R³ groups, where the naphthyl group may have one or more substituents selected from the group consisting of D, F, CN, alkyl, fluoroalkyl, hydrocarbon aryl, heteroaryl, silyl, germyl, deuterated alkyl, deuterated partially-fluorinated alkyl, deuterated hydrocarbon aryl, deuterated heteroaryl, deuterated heteroaryl deuterated silyl, and deuterated germyl.

All of the above-described embodiments for Y, Ar³, R², R³, R⁴, b, c, d, d1, and d2 in Formula IC, apply equally to Y, Ar³, R², R³, R⁴, b, c, d, d1, and d2 in Formulas IC-1 through Formula IC-9.

In some embodiments of Formula I, there are no N-containing organic groups present.

Any of the above embodiments for Formula I, Formula IA, Formula IB, Formula IC, Formula IAa, Formula IBb, and Formula ICc can be combined with one or more of the other embodiments, so long as they are not mutually exclusive. For example, the embodiment in which Ar² has Formula IA can be combined with the embodiment in which d=1 and Ar³ is naphthyl, and the embodiment in which all Y=O. The same is true for the other non-mutually-exclusive embodiments discussed above. The skilled person would understand which embodiments were mutually exclusive and would thus readily be able to determine the combinations of embodiments that are contemplated by the present application.

The compounds of Formula I can be made using any technique that will yield a C—C, C—N, C—O, C—S, or C—Si bond. A variety of such techniques are known, such as Suzuki, Yamamoto, Stille, Negishi, and metal-catalyzed C—N couplings as well as metal catalyzed and oxidative direct arylation.

Deuterated compounds can be prepared in a similar manner using deuterated precursor materials or, more generally, by treating the non-deuterated compound with deuterated solvent, such as benzene-d6, in the presence of a Bronsted or Lewis acid H/D exchange catalyst, such as trifluoromethanesulfonic acid, aluminum trichloride or ethyl aluminum dichloride. Deuteration reactions have also been described in published PCT application WO2011/053334.

Exemplary preparations are given in the Examples.

Examples of compounds having Formula I include, but are not limited to, the compounds shown below.

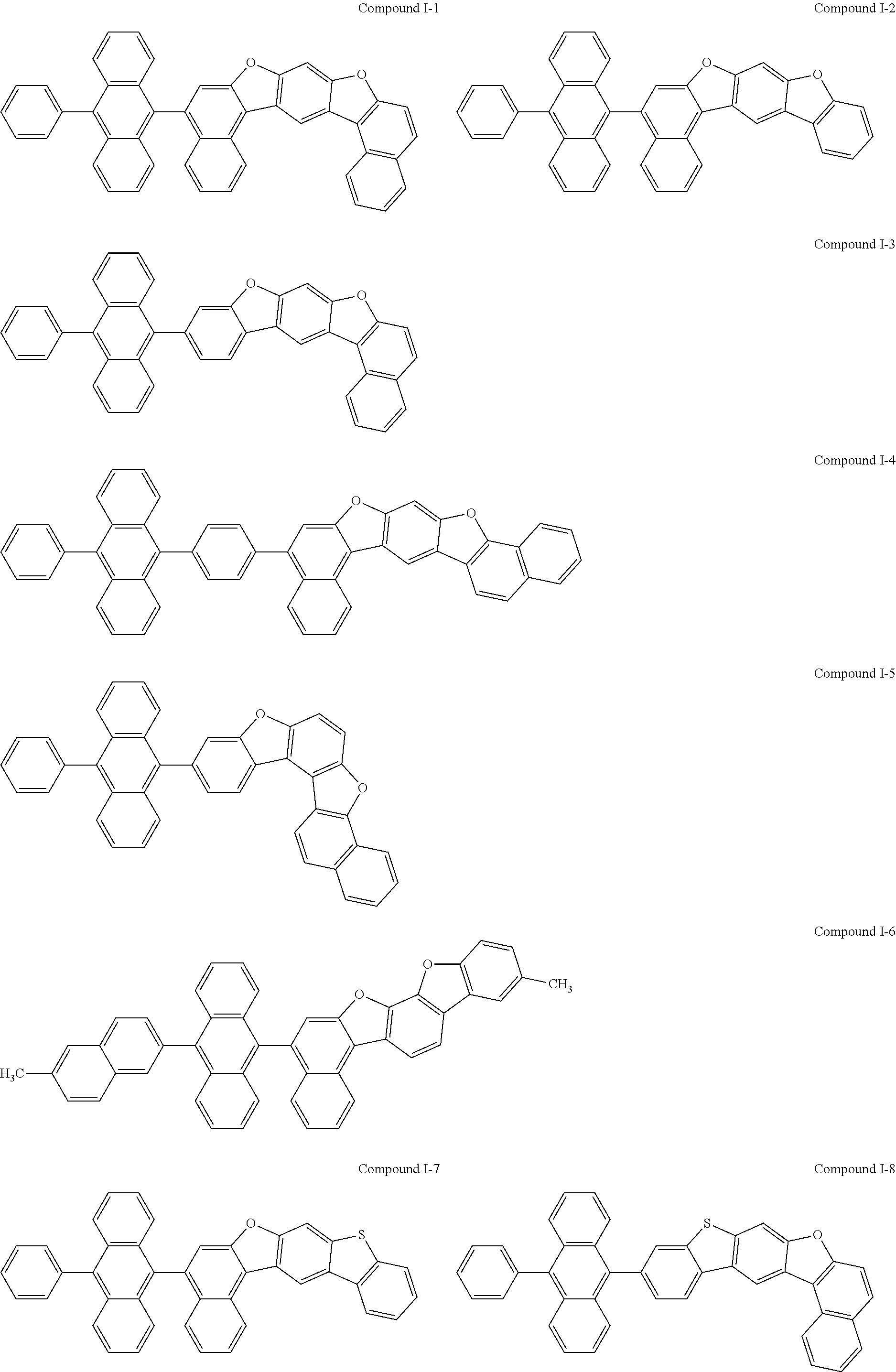

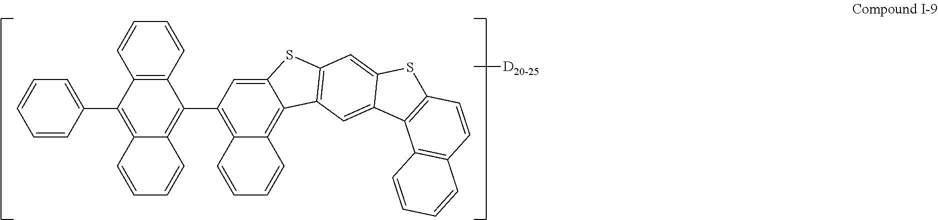

In the above structure D₂₀₋₂₅ indicates that 20-25 protons have been replaced with deuterons in unspecified locations.

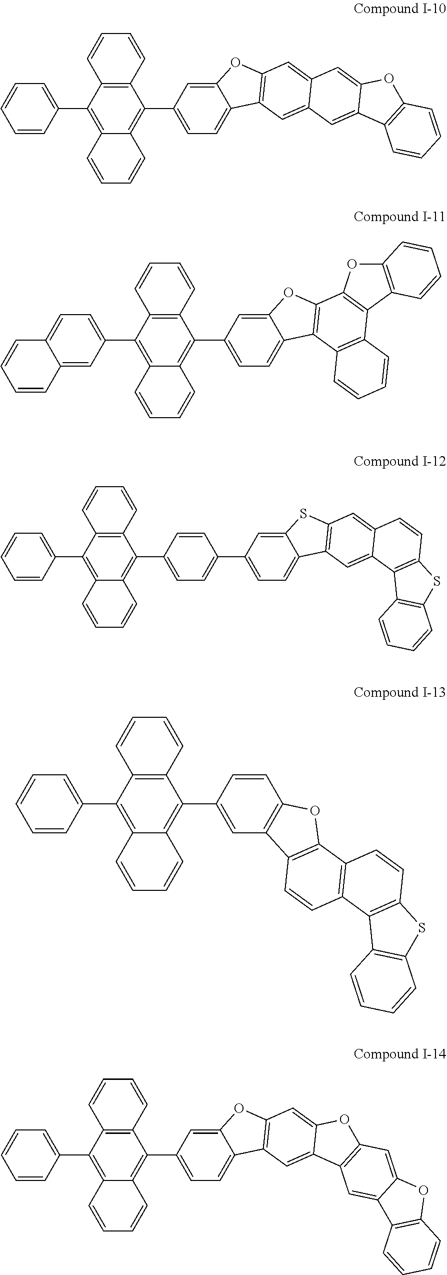 [SMILES: c1ccc(-c2c3ccccc3c(-c3ccc4c(c3)oc3cc5cc6oc7ccccc7c6cc5cc34)c3ccccc23)cc1 ; c1ccc2cc(-c3c4ccccc4c(-c4ccc5c(c4)oc4c6oc7ccccc7c6c6ccccc6c54)c4ccccc34)ccc2c1 ; c1ccc(-c2c3ccccc3c(-c3ccc(-c4ccc5c(c4)sc4cc6ccc7sc8ccccc8c7c6cc45)cc3)c3ccccc23)cc1 ; c1ccc(-c2c3ccccc3c(-c3ccc4oc5c(ccc6c5ccc5sc7ccccc7c56)c4c3)c3ccccc23)cc1 ; c1ccc(-c2c3ccccc3c(-c3ccc4c(c3)oc3cc5oc6cc7oc8ccccc8c7cc6c5cc34)c3ccccc23)cc1]

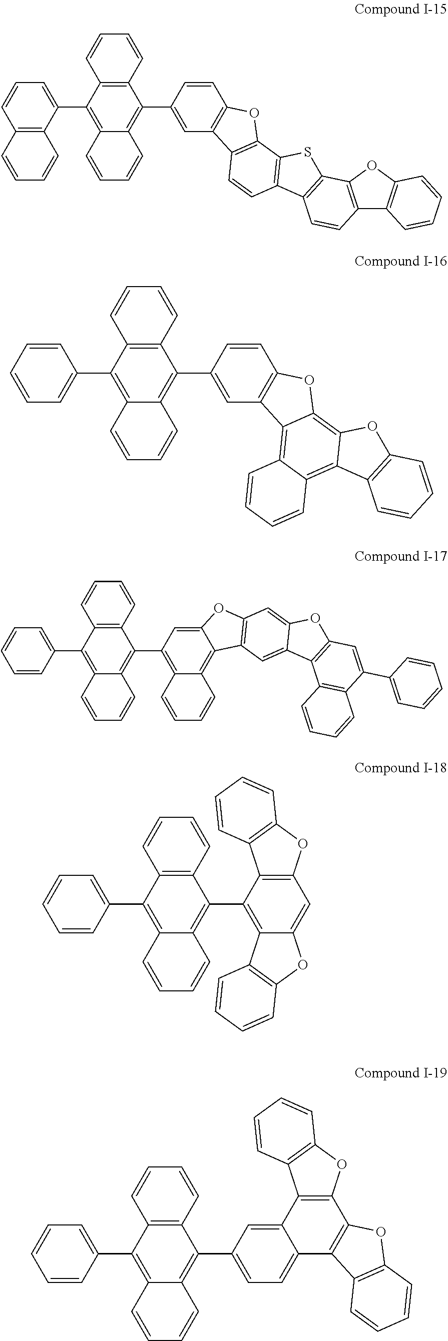 [SMILES: c1ccc2c(-c3c4ccccc4c(-c4ccc5oc6c(ccc7c8ccc9c%10ccccc%10oc9c8sc76)c5c4)c4ccccc34)cccc2c1 ; c1ccc(-c2c3ccccc3c(-c3ccc4oc5c6oc7ccccc7c6c6ccccc6c5c4c3)c3ccccc23)cc1 ; c1ccc(-c2c3ccccc3c(-c3cc4oc5cc6oc7cc(-c8ccccc8)c8ccccc8c7c6cc5c4c4ccccc34)c3ccccc23)cc1 ; c1ccc(-c2c3ccccc3c(-c3c4c(cc5oc6ccccc6c35)oc3ccccc34)c3ccccc23)cc1 ; c1ccc(-c2c3ccccc3c(-c3ccc4c(c3)c3c5ccccc5oc3c3oc5ccccc5c43)c3ccccc23)cc1]

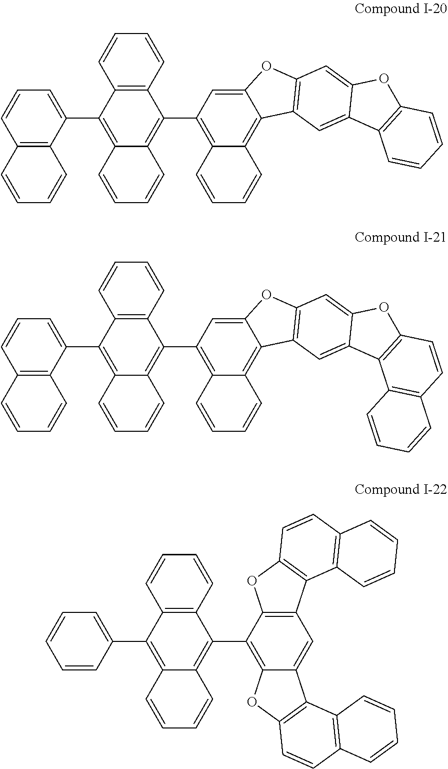 [SMILES: c1ccc2c(-c3c4ccccc4c(-c4cc5oc6cc7oc8ccccc8c7cc6c5c5ccccc45)c4ccccc34)cccc2c1 ; c1ccc2c(-c3c4ccccc4c(-c4cc5oc6cc7oc8ccc9ccccc9c8c7cc6c5c5ccccc45)c4ccccc34)cccc2c1 ; c1ccc(-c2c3ccccc3c(-c3c4oc5ccc6ccccc6c5c4cc4c3oc3ccc5ccccc5c34)c3ccccc23)cc1]

### 2. Devices

Organic electronic devices that may benefit from having one or more layers comprising the compounds having Formula I described herein include, but are not limited to, (1) devices that convert electrical energy into radiation (e.g., a light-emitting diode, light emitting diode display, diode laser, or lighting panel), (2) devices that detect a signal using an electronic process (e.g., a photodetector, a photoconductive cell, a photoresistor, a photoswitch, a phototransistor, a phototube, an infrared (“IR”) detector, or a biosensors), (3) devices that convert radiation into electrical energy (e.g., a photovoltaic device or solar cell), (4) devices that convert light of one wavelength to light of a longer wavelength, (e.g., a down-converting phosphor device); (5) devices that include one or more electronic components that include one or more organic semiconductor layers (e.g., a transistor or diode), or any combination of devices in items (1) through (5).

In some embodiments, the device includes a photoactive layer having a compound of Formula I.

In some embodiments, the device includes an anode and a cathode with a photoactive layer therebetween, where the photoactive layer includes a compound having Formula I.

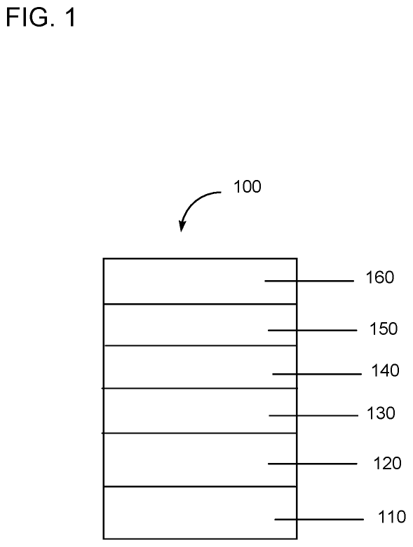

One illustration of an organic electronic device structure is shown in FIG. 1. The device 100 has a first electrical contact layer, an anode layer 110 and a second electrical contact layer, a cathode layer 160, and a photoactive layer (“EML”) 140 between them. Adjacent to the anode is a hole injection layer (“HIL”) 120. Adjacent to the hole injection layer is a hole transport layer (“HTL”) 130, comprising hole transport material. Adjacent to the cathode may be an electron transport layer (“ETL”) 150, comprising an electron transport material. As an option, devices may use one or more additional hole injection or hole transport layers (not shown) next to the anode 110 and/or one or more additional electron injection layer (“EIL”) or electron transport layer (not shown) next to the cathode 160. As a further option, devices may have an anti-quenching layer (not shown) between the photoactive layer 140 and the electron transport layer 150.

Layers 120 through 150, and any additional layers between them, are individually and collectively referred to as the active layers.

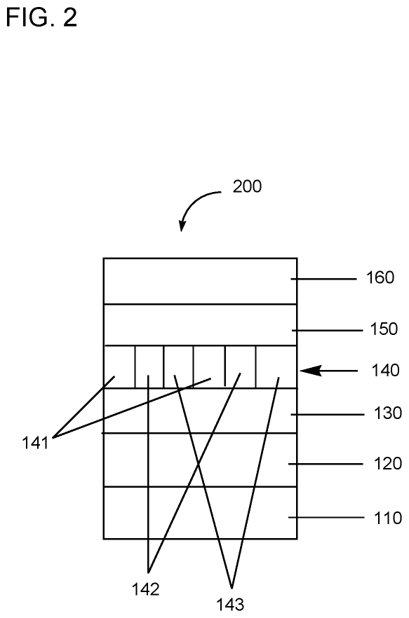

In some embodiments, the photoactive layer is pixellated, as shown in FIG. 2. In device 200, layer 140 is divided into pixel or subpixel units 141, 142, and 143 which are repeated over the layer. Each of the pixel or subpixel units represents a different color. In some embodiments, the subpixel units are for red, green, and blue. Although three subpixel units are shown in the figure, two or more than three may be used.

In some embodiments, the different layers have the following range of thicknesses: anode 110, 50-500 nm, in some embodiments, 100-200 nm; hole injection layer 120, 5-200 nm, in some embodiments, 20-100 nm; hole transport layer 130, 5-200 nm, in some embodiments, 20-100 nm; photoactive layer 140, 1-200 nm, in some embodiments, 10-100 nm; electron transport layer 150, 5-200 nm, in some embodiments, 10-100 nm; cathode 160, 20-1000 nm, in some embodiments, 30-500 nm. The location of the electron-hole recombination zone in the device, and thus the emission spectrum of the device, can be affected by the relative thickness of each layer. The desired ratio of layer thicknesses will depend on the exact nature of the materials used.

In some embodiments, the compounds having Formula I are useful as the emissive material in photoactive layer 140, having blue emission color. They can be used alone or as a dopant in a host material.

In some embodiments, the compounds having Formula I are useful as the host material in photoactive layer 140.

a. Photoactive Layer

In some embodiments, the photoactive layer includes a host material and a compound having Formula I as a dopant. In some embodiments, a second host material is present.

In some embodiments, the photoactive layer includes only a host material and a compound having Formula I as a dopant. In some embodiments, minor amounts of other materials, are present so long as they do not significantly change the function of the layer.

In some embodiments, the photoactive layer includes a dopant and a compound having Formula I as host. In some embodiments, a second host material is present. In some embodiments, more than one dopant is present.

Compounds having Formula I can be used as hosts with a variety of dopants and will perform in a similar way. Dopants are well known and broadly disclosed in the patent literature and technical journals.

Exemplary dopants include, but are not limited to, anthracenes, benzanthracenes, benz[de]anthracenes, chrysenes, pyrenes, triphenylenes, benzofluorenes, other polycyclic aromatics, and analogs having one or more heteroatoms. Exemplary dopants also include, but are not limited to, benzofurans, dibenzofurans, carbazoles, benzocarbazoles, carbazolocarbazoles, and azaborines. In some embodiments, the dopants have one or more diarylamino substituents. Dopants have been disclosed in, for example, U.S. Pat. No. 7,816,017, U.S. Pat. No. 8,465,848, U.S. Pat. No. 9,112,157, US 2006/0127698, US 2010/0032658, US 2018/0069182, US 2019/0058124, CA 3107010, EP 3109253, WO 2019003615, and WO 2019035268.

In some embodiments, the photoactive layer includes a blue luminescent material as dopant and a compound having Formula I as host.

In some embodiments, the photoactive layer includes only a dopant material and a compound having Formula I as host. In some embodiments, minor amounts of other materials are present, so long as they do not significantly change the function of the layer.

In some embodiments, the photoactive layer includes only a dopant material, a compound having Formula I as host, and a second host material. In some embodiments, minor amounts of other materials are present, so long as they do not significantly change the function of the layer.

The weight ratio of total dopant to total host material is in the range of 2:98 to 70:30; in some embodiments, 5:95 to 70:30; in some embodiments, 10:90 to 20:80.

In some embodiments, the second host material is selected from the group consisting of anthracenes, chrysenes, pyrenes, phenanthrenes, triphenylenes, phenanthrolines, naphthalenes, triazines, quinolines, isoquinolines, quinoxalines, phenylpyridines, benzodifurans, metal quinolinate complexes, indolocarbazoles, substituted derivatives thereof, and combinations thereof.

Any of the compounds of Formula I represented by the embodiments, specific embodiments, specific examples, and combination of embodiments discussed above can be used in the photoactive layer.

b. Other Device Layers

The other layers in the device can be made of any materials which are known to be useful in such layers.

The anode 110 is an electrode that is particularly efficient for injecting positive charge carriers. It can be made of, for example materials containing a metal, mixed metal, alloy, metal oxide or mixed-metal oxide, or it can be a conducting polymer, and mixtures thereof. Suitable metals include the Group 11 metals, the metals in Groups 4, 5, and 6, and the Group 8-10 transition metals. If the anode is to be light-transmitting, mixed-metal oxides of Groups 12, 13 and 14 metals, such as indium-tin-oxide, are generally used. The anode may also be made of an organic material such as polyaniline as described in “Flexible light-emitting diodes made from soluble conducting polymer,” Nature vol. 357, pp 477 479 (11 Jun. 1992). At least one of the anode and cathode should be at least partially transparent to allow the generated light to be observed.

The hole injection layer 120 includes hole injection material and may have one or more functions in an organic electronic device, including but not limited to, planarization of the underlying layer, charge transport and/or charge injection properties, scavenging of impurities such as oxygen or metal ions, and other aspects to facilitate or to improve the performance of the organic electronic device. The hole injection layer can be formed with polymeric materials, such as polyaniline (PANI) or polyethylenedioxythiophene (PEDOT), which are often doped with protonic acids. The protonic acids can be, for example, poly(styrenesulfonic acid), poly(2-acrylamido-2-methyl-1-propanesulfonic acid), and the like.

The hole injection layer can include charge transfer compounds, and the like, such as copper phthalocyanine, 1,4,5,8,9,12-hexaazatriphenylenehexacarbonitrile (HAT-CN), and the tetrathiafulvalene-tetracyanoquinodimethane system (TTF-TCNQ).

In some embodiments, the hole injection layer includes at least one electrically conductive polymer and at least one fluorinated acid polymer.

Examples of hole transport materials for layer 130 have been summarized for example, in Kirk-Othmer Encyclopedia of Chemical Technology, Fourth Edition, Vol. 18, p. 837-860, 1996, by Y. Wang. Both hole transporting molecules and polymers can be used. Commonly used hole transporting molecules are: N,N′-diphenyl-N,N′-bis(3-methylphenyl)-[1,1′-biphenyl]-4,4′-diamine (TPD), 1,1-bis[(di-4-tolylamino) phenyl]cyclohexane (TAPC), N,N′-bis(4-methylphenyl)-N,N′-bis(4-ethylphenyl)-[1,1′-(3,3′-dimethyl)biphenyl]-4,4′-diamine (ETPD), tetrakis-(3-methylphenyl)-N,N,N′,N′-2,5-phenylenediamine (PDA), a-phenyl-4-N,N-diphenylaminostyrene (TPS), p-(diethylamino)benzaldehyde diphenylhydrazone (DEH), triphenylamine (TPA), bis[4-(N,N-diethylamino)-2-methylphenyl](4-methylphenyl)methane (MPMP), 1-phenyl-3-[p-(diethylamino)styryl]-5-[p-(diethylamino)phenyl] pyrazoline (PPR or DEASP), 1,2-trans-bis(9H-carbazol-9-yl)cyclobutane (DCZB), N,N,N′,N′-tetrakis(4-methylphenyl)-(1,1′-biphenyl)-4,4′-diamine (TTB), N,N′-bis(naphthalen-1-yl)-N,N′-bis-(phenyl)benzidine (α-NPB), and porphyrinic compounds, such as copper phthalocyanine. In some embodiments, the hole transport layer includes a hole transport polymer. In some embodiments, the hole transport polymer is a distyrylaryl compound. In some embodiments, the aryl group has two or more fused aromatic rings. In some embodiments, the aryl group is an acene. The term “acene” as used herein refers to a hydrocarbon parent component that contains two or more ortho-fused benzene rings in a straight linear arrangement. Other commonly used hole transporting polymers are polyvinylcarbazole, (phenylmethyl)-polysilane, and polyaniline. It is also possible to obtain hole transporting polymers by doping hole transporting molecules such as those mentioned above into polymers such as polystyrene and polycarbonate. In some cases, triarylamine polymers are used, especially triarylamine-fluorene copolymers. In some cases, the polymers and copolymers are crosslinkable.

In some embodiments, the hole transport layer further includes a p-dopant. In some embodiments, the hole transport layer is doped with a p-dopant. Examples of p-dopants include, but are not limited to, tetrafluorotetracyanoquinodimethane (F4-TCNQ) and perylene-3,4,9,10-tetracarboxylic-3,4,9,10-dianhydride (PTCDA).

In some embodiments, more than one hole transport layer is present (not shown).

Examples of electron transport materials which can be used for layer 150 include, but are not limited to, metal chelated oxinoid compounds, including metal quinolate derivatives such as tris(8-hydroxyquinolato)aluminum (AlQ), bis(2-methyl-8-quinolinolato)(p-phenylphenolato) aluminum (BAlq), tetrakis-(8-hydroxyquinolato)hafnium (HfQ) and tetrakis-(8-hydroxyquinolato)zirconium (ZrQ); and azole compounds such as 2-(4-biphenylyl)-5-(4-t-butylphenyl)-1,3,4-oxadiazole (PBD), 3-(4-biphenylyl)-4-phenyl-5-(4-t-butylphenyl)-1,2,4-triazole (TAZ), and 1,3,5-tri(phenyl-2-benzimidazole)benzene (TPBI); quinoxaline derivatives such as 2,3-bis(4-fluorophenyl)quinoxaline; fluoranthene derivatives, such as 3-(4-(4-methylstyryl)phenyl-p-tolylamino)fluoranthene; phenanthrolines such as 4,7-diphenyl-1,10-phenanthroline (DPA) and 2,9-dimethyl-4,7-diphenyl-1,10-phenanthroline (DDPA); and mixtures thereof. In some embodiments, the electron transport layer further includes an n-dopant. N-dopant materials are well known. The n-dopants include, but are not limited to, Group 1 and 2 metals; Group 1 and 2 metal salts, such as LiF, CsF, and Cs₂CO₃; Group 1 and 2 metal organic compounds, such as Li quinolate; and molecular n-dopants, such as leuco dyes, metal complexes, such as W₂(hpp)₄ where hpp=1,3,4,6,7,8-hexahydro-2H-pyrimido-[1,2-a]-pyrimidine and cobaltocene, tetrathianaphthacene, bis(ethylenedithio)tetrathiafulvalene, heterocyclic radicals or diradicals, and the dimers, oligomers, polymers, dispiro compounds and polycycles of heterocyclic radical or diradicals.

In some embodiments, an anti-quenching layer may be present between the photoactive layer and the electron transport layer to prevent quenching of blue luminance by the electron transport layer. To prevent energy transfer quenching, the singlet energy of the anti-quenching material has to be higher than the singlet energy of the blue emitter. To prevent electron transfer quenching, the LUMO level of the anti-quenching material has to be shallow enough (with respect to the vacuum level) such that electron transfer between the emitter exciton and the anti-quenching material is endothermic. Furthermore, the HOMO level of the anti-quenching material has to be deep enough (with respect to the vacuum level) such that electron transfer between the emitter exciton and the anti-quenching material is endothermic. In general, anti-quenching material is a large band-gap material with high singlet and triplet energies.

The cathode 160, is an electrode that is particularly efficient for injecting electrons or negative charge carriers. The cathode can be any metal or nonmetal having a lower work function than the anode. Materials for the cathode can be selected from alkali metals of Group 1 (e.g., Li, Cs), the Group 2 (alkaline earth) metals, the Group 12 metals, including the rare earth elements and lanthanides, and the actinides. Materials such as aluminum, indium, calcium, barium, samarium and magnesium, as well as combinations, can be used.

Alkali metal-containing inorganic compounds, such as LiF, CsF, Cs₂O and Li₂O, or Li-containing organometallic compounds can also be deposited between the organic layer 150 and the cathode layer 160 to lower the operating voltage. This layer, not shown, may be referred to as an electron injection layer.

It is known to have other layers in organic electronic devices. For example, there can be a layer (not shown) between the anode 110 and hole injection layer 120 to control the amount of positive charge injected and/or to provide band-gap matching of the layers, or to function as a protective layer. Layers that are known in the art can be used, such as copper phthalocyanine, silicon oxy-nitride, fluorocarbons, silanes, or an ultra-thin layer of a metal, such as Pt. Alternatively, some or all of anode layer 110, active layers 120, 130, 140, and 150, or cathode layer 160, can be surface-treated to increase charge carrier transport efficiency. The choice of materials for each of the component layers is preferably determined by balancing the positive and negative charges in the emitter layer to provide a device with high electroluminescence efficiency.

It is understood that each functional layer can be made up of more than one layer.

c. Device Fabrication

The device layers can be formed by any deposition technique, or combinations of techniques, including vapor deposition, liquid deposition, and thermal transfer.

In some embodiments, the device is fabricated by liquid deposition of the hole injection layer, the hole transport layer, and the photoactive layer, and by vapor deposition of the anode, the electron transport layer, an electron injection layer and the cathode. Suitable liquid deposition techniques are well known in the art.

In some embodiments, all the device layers are fabricated by vapor deposition. Such techniques are well known in the art.

### EXAMPLES

The concepts described herein will be further described in the following examples, which do not limit the scope of the invention described in the claims.

### Synthesis Examples

These examples illustrate the preparation of compounds having Formula I, as described above.

### Synthesis Example 1

This example illustrates the preparation of a compound having Formula I, Compound I-1.

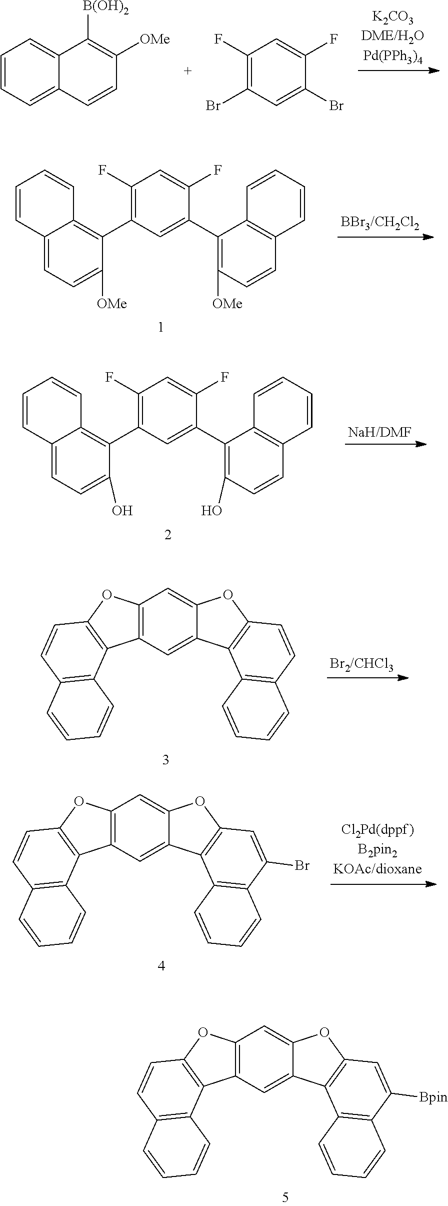

(a) 1,1′-(4,6-Difluoro-1,3-phenylene)bis[2-methoxy-naphthalene] (1).

A mixture of 2-(2-methoxy-1-naphthalenyl)-boronic acid (32.79 g, 162 mmole, 2.2 equivalents), 1,5-dibromo-2,4-difluorobenzene (20.02 g, 73.64 mmole, 1 equivalent), potassium carbonate (44.79 g, 324 mmole, 5 equivalents), Pd(PPh₃)₄ (3.74 g, 3.24 mmole, 4.4 mol %), 1,2-dimethoxyethane (600 ml), water (150 ml) purged with nitrogen, stirred at reflux for 19 hours under nitrogen atmosphere. Reaction mixture cooled down, precipitated product (isomer-1) collected by filtration, washed with water (200 ml), methanol (200 ml). Combined filtrate formed new precipitate (isomer-2) that was filtered, washed with water and methanol. Precipitates combined, dried in vacuum to give 26.7 g of crude product 1 that was used for the next step without further purification. For analytical purposes atropoisomers could be separated by column chromatography on silica gel using gradient eluation with mixtures of hexanes and dichloromethane. MS: MH+=427. ¹H-NMR of diastereomer 1 (CDCl₃, 500 MHz): 3.95 (s, 6H), 7.14 (t, 1H, J=9 Hz), 7.34-7.37 (m, 3H), 7.38 (d, 2H, J=9 Hz), 7.43 (td, 2H, J1=1 Hz, J2=8 Hz), 7.68 (d, 2H, J=9 Hz), 7.83 (d, 2H, J=8 Hz), 7.92 (d , 2H, J=9 Hz). ¹H-NMR of diastereomer 2 (CDCl₃, 500 MHz): 3.91 (s, 6H), 7.14 (t, 1H, J=9 Hz), 7.31-7.38 (m, 3H), 7.38 (d, 2H, J=9 Hz), 7.44 (td, 2H, J1=1 Hz, J2=8 Hz), 7.64 (d, 2H, J=9 Hz), 7.83 (d, 2H, J=9 Hz), 7.92 (d, 2H, J=9 Hz).

(b) 1,1′-(4,6-Difluoro-1,3-phenylene)bis-(2-naphthalenol) (2).

Starting compound 1 (8.83 g), suspended in 70 ml of dichloromethane under nitrogen atmosphere followed by addition of 62 ml of 1M solution of BBr₃ (3 equivalents) in dichloromethane and resulting solution left to stir at ambient temperature overnight. After that reaction mixture poured into ice and stirred for 7 hours. Organic phase separated, aqueous phase extracted with dichloromethane, dichloromethane evaporated using rotary evaporator, the residue redissolved in toluene and dichloromethane followed by evaporation and drying using rotary evaporator for 30 min and in vacuum overnight. Yield—quantitative. Crude product 2 was used for the next step without further purification. MS: MH+=399. ¹H-NMR, mixture of diastereomers, (CDCl₃, 500 MHz): 7.21-7.26 (m, 4H), 7.28-7.33 (m, 1H), 7.36-7.39 (m, 2H), 7.44-7.53 (m, 5H), 7.82-7.86 (m, 4H).

(c) Dinaphtho[1,2-d:1′,2′-d′]benzo[1,2-b:5,4-b′]difuran (3).

1,1′-(4,5-Difluoro-1,3-phenylene)bis(2-naphthalenol) 2 (8.25 g, 20.71 mmole) was dissolved in dry dimethylformamide (100 ml) under nitrogen atmosphere followed by addition of sodium hydride (60% suspension in mineral oil, 4.14 g, 103.6 mmole, 5 equivalents) at once. Resulting mixture was stirred at 150° C. for overnight. After that the mixture was cooled down, carefully diluted with methanol (100 ml), precipitate collected by filtration, washed with water, methanol, dried in vacuum to give 2.87 g of dinaphtho[1,2-d:1′,2′-d′]benzo[1,2-b:5,4-b′]difuran 3. ¹H-NMR (CDCl₃, 500 MHz): 7.64 (t, 2H, J=8 Hz), 7.84 (d, 2H, J=9 Hz), 7.90 (t, 2H, J=8 Hz), 7.98 (d, 2H, J=9 Hz), 7.99 (s, 1H), 8.10 (d, 2H, J=9 Hz), 8.89 (d, 2H, J=9 Hz), 9.31 (s, 1H). MS: MH+=359. UV-vis in acetonitrile—water, λₘₐₓ, nm: 359, 261, 237. Emission in toluene: 368 nm.

(d) 5-Bromo-dinaphtho[1,2-d:1′,2′-d′]benzo[1,2-b:5,4-b′]difuran (4).

Bromine (11.35 g, 71.04 mmole, 1.9 equivalents) was slowly added via dropping funnel over period of 4 hours to a stirred suspension of compound 3 (13.4 g, 37.4 mmole) in 700 ml of chloroform at 70 C. Reaction mixture stirred at ambient temperature for overnight and at 70 C for an additional hour, hot solution filtered, washed with small amount of dichloromethane, dried in vacuum to give 14.03 g of the product. Filtrate diluted with 400 ml of ethanol, precipitate collected by filtration to give approx. 0.4 g of the product. Initial filtrate evaporated to volume approx. 50 ml, precipitate filtered, dried to give additional amount (0.5 g) of a mixture of mono and bis-bromo products. The mixture of mono- and bis-brominated compounds was used for the next step without further purification. Data for monobromominated product 4: ¹H-NMR (CDCl₃, 500 MHz): 7.64 (t, 1H, J=8 Hz), 7.74 (t, 1H, J=8 Hz), 7.84 (d, 1H, J=9 Hz), 7.90 (t, 1H, J=8 Hz), 7.95 (t, 1H, J=8 Hz), 7.97-8.00 (m, 2H), 8.10 (d, 1H, J=8 Hz), 8.20 (s, 1H), 8.52 (d, 1H, J=9 Hz), 8.86 (d, 1H, J=8 Hz), 8.89 (d, 1H, J=8 Hz), 9.29 (s, 1H).

(e) 5-(4,4,5,5-Tetramethyl-1,3,2-dioxaborolan-2-yl)-dinaphtho[1,2-d:1′,2′-d′]benzo[1,2-b:5,4-b′]difuran (5).

A mixture of the above mono-4 and bis-brominated compounds (14.51 g, 33.18 mmole), bis(pinacolato)diboron (12.64 g, 49.77 mmole), potassium acetate (16.28 g, 166 mmole, 5 equivalents), (1,1′-bis(diphenylphosphino)ferrocene)palladium(II) dichloride (2.426 g, 3.318 mmole), 1,4-dioxane (500 ml) was heated at 100 C with stirring under nitrogen atmosphere for 20 hours. Reaction mixture cooled down, passed through a filter filled with celite eluating with dichloromethane, solvents evaporated using rotary evaporator, the residue dissolved in dichloromethane, evaporated onto celite and subjected to chromatography purification on silica gel column using gradient eluation with mixtures of hexanes and dichloromethane. Fractions containing monoborylated product combined, eluent evaporated, the residue dried in vacuum to give 4.04 g of monoborylated product 5. Bis-borylated product (3.89 g) could also be isolated. Data for monoborylated product 5: ¹H-NMR (CDCl₃, 500 MHz): 1.51 (s, 12H), 7.59 (t, 1H, J=8 Hz), 7.66 (t, 1H, J=8 Hz), 7.76 (d, 1H, J=9 Hz), 7.82-7.86 (m, 2H), 7.90 (s, 1H), 7.91 (d, 1H, J=8 Hz), 8.04 (d, 1H, J=8 Hz), 8.40 (s, 1H), 8.77 (d, 1H, J=8 Hz), 8.82 (d, 1H, J=8 Hz), 9.03 (d, 1H, J=8 Hz), 9.20 (s, 1H). Data for bisborylated product: ¹H-NMR (CDCl₃, 500 MHz): 1.51 (s, 24 H), 7.65 (t, 2H, J=8 Hz), 7.84 (t, 2H, J=8 Hz), 7.92 (s, 1H), 8.41 (s, 2H), 8.83 (d, 2H, J=8 Hz), 9.03 (d, 2H, J=9 Hz), 9.26 (s, 1H).

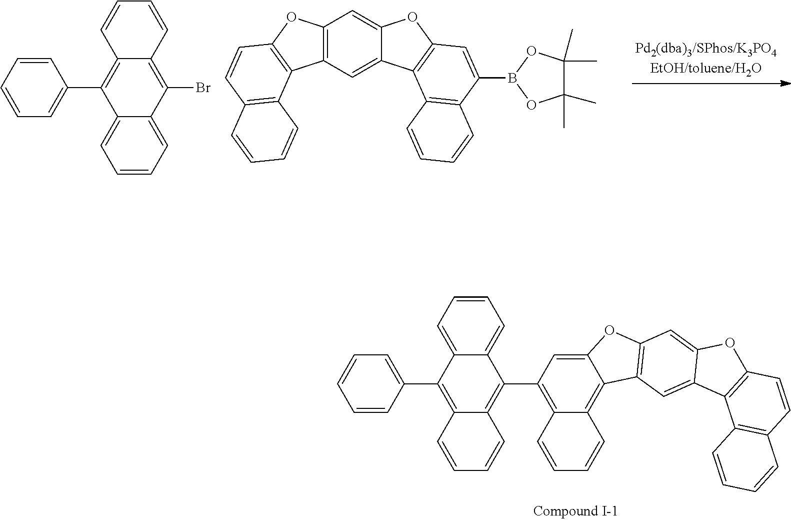

(f) 5-(10-Phenyl-anthracene-9-yl)-dinaphtho[1,2-d:1′,2′-d′]benzo[1,2-b:5,4-b′]difuran (Compound I-1). A mixture of 9-bromo-10-phenylanthracene (2.779 g, 8.34 mmole), 5-(4,4,5,5-tetramethyl-1,3,2-dioxaborolan-2-yl)-dinaphtho[1,2-d:1′,2′-d′]benzo[1,2-b:5,4-b′]difuran 5 (4.04 g, 8.34 mmole), Pd2(dba)3 (144 mg, 0.157 mmole), SPhos (516 mg, 1.256 mmole), potassium phosphate (5.0 g, 23.55 mmole), toluene (200 ml), water (40 ml), ethanol (80 ml) was purged with nitrogen and heated at 100 C with stirring under nitrogen atmosphere for 16 hours. Reaction mixture cooled down, precipitate filtered, washed with toluene, water, methanol, dried in vacuum to give 4.46 g of crude product. The product was dissolved in hot chloroform (2 L), passed through a filter filled with silica gel, florisil eluating with hot chloroform. Chloroform evaporated minimal volume fractionally collecting precipitates to give 4.09 g of the product with purity greater than 98% by UPLC. The product was subsequently dissolved in 170 ml of 1,2-dichlorobenzene at 180 C. Precipitate collected by filtration, dried in vacuum at 90 C to give 2.855 g of product, Compound I-1, with purity 99.50% by UPLC. MS: MH+611. ¹H-NMR (CDCl₃, 500 MHz): 7.25-7.28 (m, 3H), 7.34-7.38 (m, 3H), 7.43 (d, 1H, J=8 Hz), 7.53-7.66 (m, 4 H), 7.65-7.68 (m, 3H), 7.80 (br s, 2H, J=8 Hz), 7.88 (d, 1H, J=9 Hz), 7.90 (t, 1H, J=8 Hz), 7.95 (t, 1H, J=8 Hz), 7.98 (s, 1H), 8.02 (d, 1H, J=9 Hz), 8.06 (s, 1H), 8.13 (d, 1H, J=8 Hz), 8.97 (d, 1H, J=8 Hz), 9.06 (d, 1H, J=9 Hz), 9.46 (s, 1H).

### Synthesis Example 2

This example illustrates the preparation of a compound having

Formula I, Compound I-16.

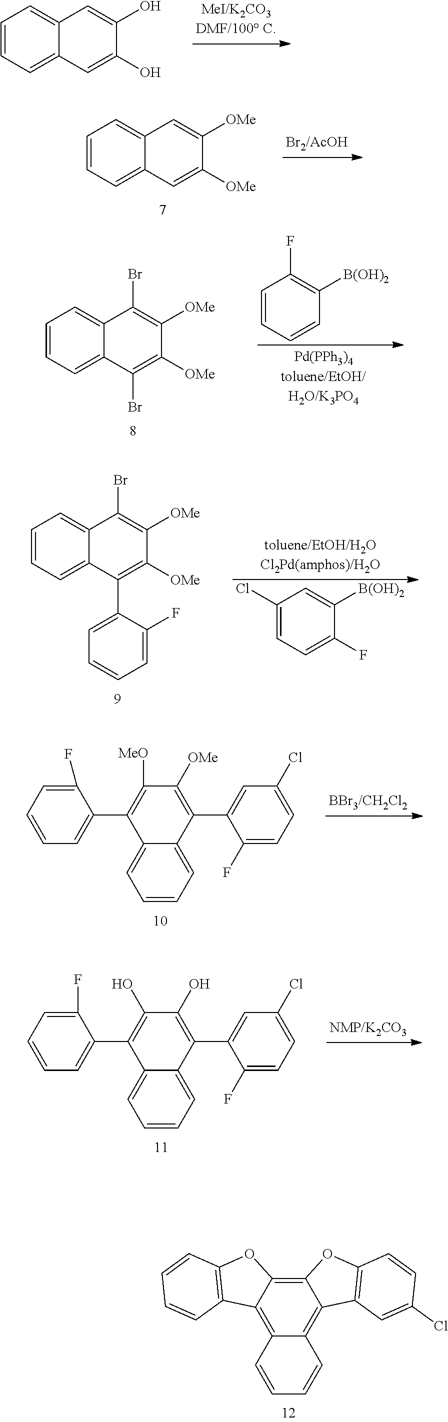

### (a) 2,3-Dimethoxynaphthalene (7).

A mixture of 2,3-dihydroxynaphthalene (25 g, 156.08 mmole), methyl iodide (66.46 g, 468.24 mmole), potassium carbonate (64.72 g, 468.24 mmole) in dimethylformamide (100 ml) was heated under nitrogen atmosphere at 100 C for 18 hours with stirring. Reaction mixture cooled down, diluted with approx. 250 ml of water, precipitate collected, washed with water. Filtrate filtered again, precipitate washed with water. Combined precipitates dried in vacuum to give 24.4 g (83%) of the product 7.

(b) 1,4-Dibromo-2,3-dimethoxynaphthalene (8).

To a suspension of 2,3-dimethoxynaphthalene (19.07 g, 101.32 mmole) in 140 ml of acetic acid was added dropwise a solution of bromine (32.23 g, 201.68 mmole) in acetic acid (60 ml) over period of 20 min maintaining internal temperature below 30 C. Resulting solution stirred under ambient temperature for 2 hours. Reaction mixture quenched with acetone, poured into approx. 500 ml of water, solids decanted, treated with hexanes, precipitate collected by filtration, filtrate evaporated to minimal volume, filtered again, dried in vacuum to give 5.5 g of the product. Aqueous phase extracted with dichloromethane, combined with filtrate and subjected to column chromatography on silica gel column using gradient eluation with mixtures of hexanes—dichloromethane. Fractions containing product combined, eluent evaporated, solids dried in vacuum to give additionally 9.15 g of the product. ¹H-NMR (CDCl₃, 500 MHz): 4.01 (s, 6H), 7.55-7.59 (m, 2H), 8.23-8.26 (m, 2H).

(c) 1-Bromo-4-(2-fluorophenyl)-2,3-dimethoxynaphthalene (9).

A mixture of 1,4-dibromo-2,3-dimethoxynaphthalene 8 (11.41 g, 32.98 mmole), 2-fluorophenylboronic acid (6.92 g, 49.47 mmole, 1.5 equivalents), Pd(PPh₃)₄ (3.81 g, 3.298 mmole, 10mol %), potassium phosphate (28 g, 131.9 mmole), toluene (100 ml), water (20 ml), ethanol (40 ml) was purged with nitrogen and heated at 100 C with stirring under nitrogen atmosphere for 7 hours. Reaction mixture cooled down, diluted with 100 ml of water, toluene layer separated. Toluene distilled off using rotary evaporator, the residue redissolved in dichloromethane, absorbed onto celite, subjected to chromatography on silica gel column using gradient eluation with mixtures of hexanes and dichloromethane. Fractions containing recovered starting material combined, eluent evaporated, dried in vacuum to give 4.2 g of recovered compound 8. Fractions containing monocoupled product combined, eluent evaporated, the residue dried in vacuum to give 5.08 g of 1-bromo-4-(2-fluorophenyl)-2,3-dimethoxynaphthalene 9. ¹H-NMR (CDCl₃, 500 MHz): 3.77 (s, 3H), 4.03 (s, 3H), 7.24-7.42 (m, 5 H), 7.46-7.54 (m, 2H), 8.28 *d, 1H, J=8 Hz).

(d) 1-(5-Chloro-2-fluorophenyl)-4-(2-fluorophenyl)-2,3-dimethoxynaphthalene (10).

A mixture of 1-bromo-4-(2-fluorophenyl)-2,3-dimethoxynaphthalene 9 (1.503 g, 4.16 mmole), 5-chloro-2-fluorophenylboronic acid (1.16 g, 6.6 mmole, 1.6 equivalents), Cl₂Pd(amphos) (100 mg, 0.142 mmole, 3 mol %), potassium carbonate (2 g, 14.5 mmole, 3.5 equivalents), toluene (50 ml), water (10 ml), ethanol (20 ml) was purged with nitrogen and heated at 100 C with stirring under nitrogen atmosphere for 50 minutes. Reaction mixture cooled down, diluted with water, toluene layer separated. Toluene distilled off using rotary evaporator, the residue redissolved in dichloromethane, absorbed onto celite, subjected to chromatography on silica gel column using gradient eluation with mixtures of hexanes and dichloromethane. Fractions containing biscoupled product combined, eluent evaporated, the residue dried in vacuum to give 1.1 g of 1-(5-chloro-2-fluorophenyl)-4-(2-fluorophenyl)-2,3-dimethoxynaphthalene 10. ¹H-NMR (CDCl₃, 500 MHz): 3.77, 3.78 and 3.82 (s, 6H, atropoisomers), 7.20-7.24 (m, 1H), 7.28-7.51 (m, 10H).

(e) 1-(5-Chloro-2-fluorophenyl)-4-(2-fluorophenyl)-2,3-naphthalenediol (11).

To a solution of compound 10 (1.1 g) in 10 ml of dichloromethane under nitrogen atmosphere was added BBr₃ (1.02 ml, 2.69 g) and the resulting solution was left to stir at ambient temperature for 1 hour. After that reaction mixture diluted with 100 ml of dichloromethane, hydrolyzed with 50 ml of water. Organic phase separated, aqueous phase extracted with dichloromethane, dichloromethane evaporated using rotary evaporator, the residue redissolved in toluene and dichloromethane followed by evaporation and drying using rotary evaporator for 30 min and in vacuum overnight. Yield—quantitative. Crude product 10 was used for the next step without further purification. ¹H-NMR, (CDCl₃, 500 MHz): 5.49 (s, 1H), 5.75 (s, 1H), 7.17-7.19 (m, 1H), 7.24-7.48 (m, 9H), 7.53-7.58 (m, 1H).

(f) 3-Chloro-naphtho[2,1-b:3,4-b′]bisbenzofuran (12).

A mixture of 1-(5-chloro-2-fluorophenyl)-4-(2-fluorophenyl)-2,3-naphthalenediol 11 (1.02 g), potassium carbonate (1.666 g) and N-methylpyrrolidinone (35 ml) was stirred with heating at 140 C under nitrogen atmosphere for 13 hours. Reaction mixture cooled down, diluted with 30 ml of water, precipitate filtered, washed with water dried in vacuum to give 0.7 g of product 12. ¹H-NMR, (CDCl₃, 500 MHz): 7.49 (dd, 1H, J1=9 Hz, J2=2 Hz), 7.53-7.59 (m, 2H), 7.71 (d, 1H, J=9 Hz), 7.75-7.79 (m, 2H), 7.80-7.82 (m, 1H), 8.38 (d, 1H, J=2 Hz), 8.44-8.46 (m, 1H), 8.62-8.65 (m, 1H), 8.74-8.77 (m, 1H).

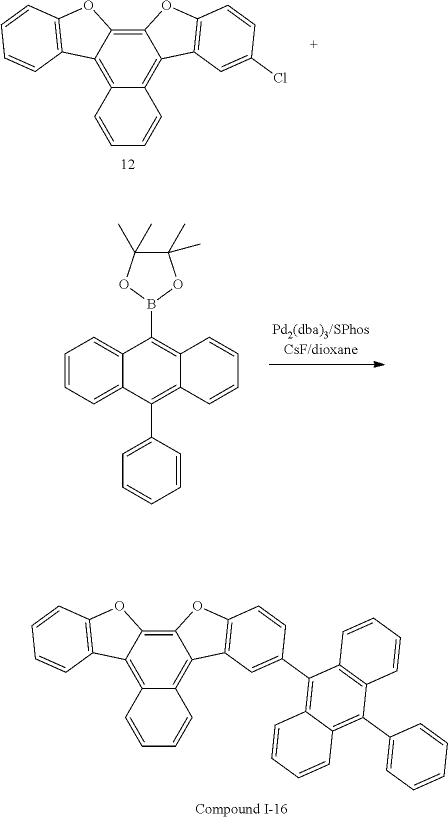 [SMILES: Clc1ccc2oc3c4oc5ccccc5c4c4ccccc4c3c2c1 ; CC1(C)OB(c2c3ccccc3c(-c3ccccc3)c3ccccc23)OC1(C)C ; c1ccc(-c2c3ccccc3c(-c3ccc4oc5c6oc7ccccc7c6c6ccccc6c5c4c3)c3ccccc23)cc1]

(g) 3-(10-Phenyl-9-anthracenyl)-naphtho[2,1-b:3,4-b′]bisbenzofuran (13).

A mixture of 4,4,5,5-tetramethyl-2-(10-phenyl-9-anthracenyl)-1,3,2-dioxaborolane (0.777 g, 2.042 mmole), 3-chloro-naphtho[2,1-b:3,4-b′]bisbenzofuran 12 (2.07 g, 2.042 mmole), Pd₂(dba)₃ (93 mg, 0.1021 mmole), SPhos (279 mg, 0.6796 mmole), cesium fluoride (1.55 g, 10.21 mmole), dioxane (175 ml) was heated at 100 C with stirring under nitrogen atmosphere for 29 hours. Reaction mixture cooled down, precipitate filtered, washed with water, dried in vacuum to give 0.78 g of crude product. Filtrate passed through a filter filled with basic alumina, florisil, silica gel eluating with dichloromethane. Filtrate evaporated to approx. 10 ml, precipitate collected by filtration to give additional amount of crude product (0.23 g). Both portions of crude product dissolved each in hot chloroform, passed through a filter filled with basic alumina, florisil and silica gel eluating with chloroform or dichloromethane. Chloroform evaporated to minimal amount, precipitates collected by filtration to give totally 0.527 g of Compound I-16 with purity greater than 99.93% by UPLC. MS: MH+561. ¹H-NMR (CD₂Cl₂, 500 MHz): 7.34 (s, 1H), 7.36-7.41 (m, 4H), 7.55-7.57 (m, 2H), 7.60-7.72 (m, 6H), 7.76-7.83 (m, 5H), 7.91-7.93 (m, 1H), 8.11 (d, 1H, J=8 Hz), 8.56 (d, 1H, J=7 Hz), 8.63 (d, 1H, J=1 Hz), 8.74 (d, 1H, J=8 Hz), 8.87 (H, J=8 Hz).

### Synthesis Example 3

This example illustrates the preparation of a compound having Formula I, Compound I-2.

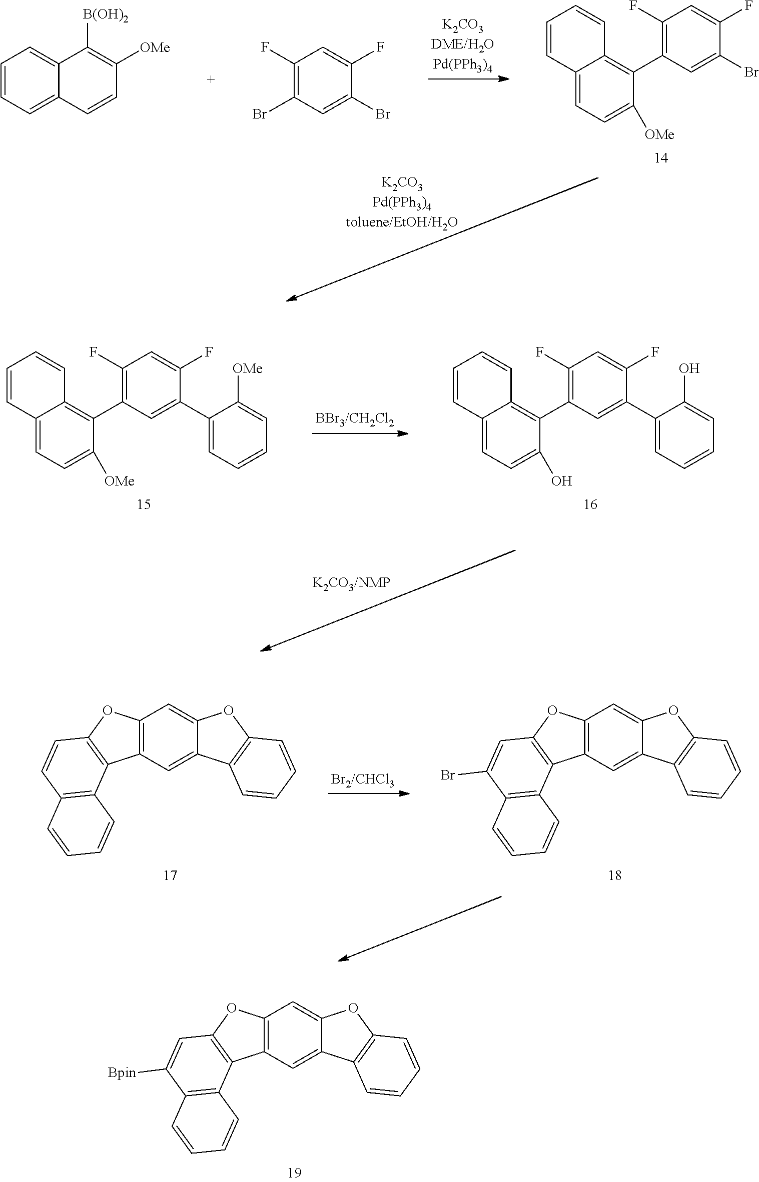

(a) 1-(5-Bromo-2,4-difluorophenyI)-2-methoxy-naphthalene (14).

A mixture of 2-(2-methoxy-1-naphthalenyl)-boronic acid (34 g, 168 mmole), 1,5-dibromo-2,4-difluorobenzene (41.2 g, 151.5 mmole), cesium fluoride (76.7 g, 505 mmole,), Pd(PPh₃)₄ (9.72 g, 8.42 mmole), dioxane (1000 ml) stirred at reflux for 6 hours under inert atmosphere. Reaction mixture cooled down, quenched with water (500 ml), extracted with ethyl acetate (3×400 ml), combined organic phase washed with brine, dried over magnesium sulfate. The residue after evaporation of solvents was subjected to chromatography purification on silica gel column using eluation with mixtures of ethyl acetate—petroleum ether to give compound 14 as white solids (average yield—46%). MS: MH+=349.

(b) 1-(4,6-Difluoro-2′-methoxy-[1,1′-biphenyl]-3-yl)-2-methoxynaphthalene (15).

A mixture of 1-(5-bromo-2,4-difluorophenyl)-2-methoxy-naphthalene 14 (100 g, 286 mmole), 2-methoxyphenylboronic acid (39.17 g, 258 mmole), cesium fluoride (130 g, 858 mmole,), Pd(PPh₃)₄ (16.5 g, 14.3 mmole), dioxane (3000 ml) stirred at reflux for 4 hours under inert atmosphere. Reaction mixture cooled down, quenched with water (1000 ml), extracted with ethyl acetate (3×600 ml), combined organic phase washed with brine, dried over magnesium sulfate. The residue after evaporation of solvents was subjected to chromatography purification on silica gel column using eluation with mixtures of ethyl acetate—petroleum ether to give compound 15 as white solids (95 g, 89%). MS: MH+=349. MS: MH+=377.

(c) 1-(4,6-Difluoro-2′-hydroxy-[1,1′-biphenyl]-3-yl)naphthalen-2-ol (16).

To a stirred solution of compound 15 (50 g, 132.6 mmole) in 1000 ml of dichloromethane under inert atmosphere cooling with ice/water bath was added 398 ml of 1M dichloromethane solution of BBr₃ (398 mmole, 3 equivalents). After that the mixture was stirred as ambient temperature for 4 hours, poured into ice, extracted with dichloromethane (2×1000 ml), combined organic phase washed with aq. sodium bicarbonate, dried over magnesium sulfate. The residue after evaporation of solvents was triturated with pentane, dried and used for the next step without further purification (average yield of compound 16—88%). MS: MH+=349.

### (d) Benzofuro[3,2-f]naphtho[2,1-b]benzofuran (17).

1-(4,6-difluoro-2′-hydroxy-[1,1′-biphenyl]-3-yl)naphthalen-2-ol 16 (15 g, 43.06 mmole) was dissolved in dry N-methylpyrrolidinone (150 ml) under inert atmosphere followed by addition of potassium carbonate (17.8 g, 129.2 mmole). Resulting mixture was stirred at 120° C. for 3 hours. After that the mixture was cooled down, poured into ice, precipitate collected by filtration, dried to give compound 17 (11 g, 83%) with purity 98.5% by UPLC. MS: MH+=309.

### (e) 5-Bromo-benzofuro[3,2-f]naphtho[2,1-b]benzofuran (18).

Solution of bromine (1.91 ml, 37.29 mmole) in chloroform (50 ml) was slowly added via dropping funnel at 0 C over period of 15 min to a stirring solution of compound 17 (11 g, 37.29 mmole) in 350 ml of chloroform. Reaction mixture stirred at ambient temperature for 5 hours. Reaction mixture was quenched with aq. sodium thiosulfate at 0 C, organic phase separated, aqueous phase extracted with dichloromethane (3×200 ml). The residue after evaporation of solvents was heated with ethanol (150 ml) for 30 min, filtered, dried to give compound 18 (10 g, 72%) with purity 94% by UPLC that was used for the next step without further purification. MS: MH+=387.

(f) 5-(4,4,5,5-Tetramethyl-1,3,2-dioxaborolan-2-yl)-benzofuro[3,2-f]naphtho[2,1-b]benzofuran (19).

A mixture of compound 18 (10 g, 28.41 mmole), bis(pinacolato)diboron (10.82 g, 42.61 mmole), potassium acetate (8.36 g, 85.23 mmole), (1,1′-bis(diphenylphosphino)ferrocene)palladium(II) dichloride (1.16 g, 1.42 mmole), 1,4-dioxane (165 ml) was heated at 100 C with stirring under inert atmosphere for 6 hours. Reaction mixture cooled down, diluted with ethyl acetate (500 ml), passed through a filter filled with celite, solvents evaporated, the residue subjected to chromatography purification on silica gel column using eluation with mixtures of petroleum ether and ethyl acetate. Fractions containing monoborylated product combined, eluent evaporated, the residue dried in vacuum to give 8 g of crude product with purity 96.9% by UPLC. The product was further purified by dissolving in chloroform (20 ml) and precipitating with pentane (100 ml) collecting precipitate by filtration, drying in vacuum to give compound 19 (6 g) with purity 99.4%. MS: MH+=435.

### (g) 5-(10-Phenyl-anthracene-9-yl)benzofuro[3,2-f]naphtho[2,1-b]benzofuran (Compound I-2).

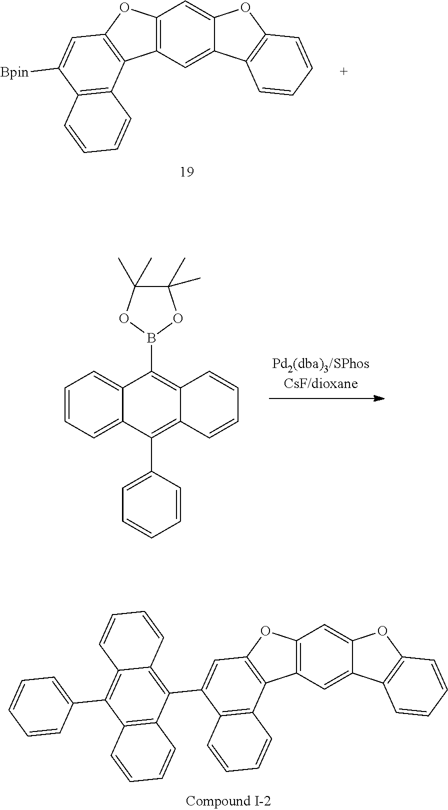

A mixture of 9-bromo-10-phenylanthracene (5.07 g, 15.2 mmole), 5-(4,4,5,5-tetramethyl-1,3,2-dioxaborolan-2-yl)benzo[d]naphtho[1,2-d′]benzo[1,2-b:5,4-b′]difuran 19 (6.61 g, 15.2 mmole), Pd2(dba)3 (278 mg, 0.304 mmole), SPhos (500 mg, 1.22 mmole), potassium phosphate (9.04 g, 42.6 mmole), toluene (364 ml), water (73 ml), ethanol (146 ml) was heated at reflux with stirring under nitrogen atmosphere for overnight. Reaction mixture cooled down, precipitate filtered, washed with toluene, hexanes, water, methanol, dried in vacuum to give 6.84 g of crude product. The product was dissolved in hot chloroform (550 ml), passed through a filter filled with silica gel, florisil and basic alumina eluating with hot chloroform. Chloroform evaporated to volume 200 ml, precipitate collected by filtration, stirred in a mixture of chloroform and methanol (1:2) for 30 min., filtered, dried in vacuum to give Compound I-2 (5.9 g) with purity greater than 99.99% by UPLC. MS: MH+561. ¹H-NMR (toluene-d₈, 500 MHz): 6.99-7.13 (m, 5H), 7.20-7.22 (m, 2H), 7.32-7.54 (m, 8H), 7.59 (d, 2H, J=9 Hz), 7.69 (s, 1H), 7.72 (s, 1H), 7.81-7.82 (m, 1H), 7.92 (d, 2H, J=9 Hz), 8.82 (s, 1H), 8.84 (d, 1H, J=9 Hz).

### Synthesis Example 4

This example illustrates the preparation of a compound having Formula I, Compound I-20.

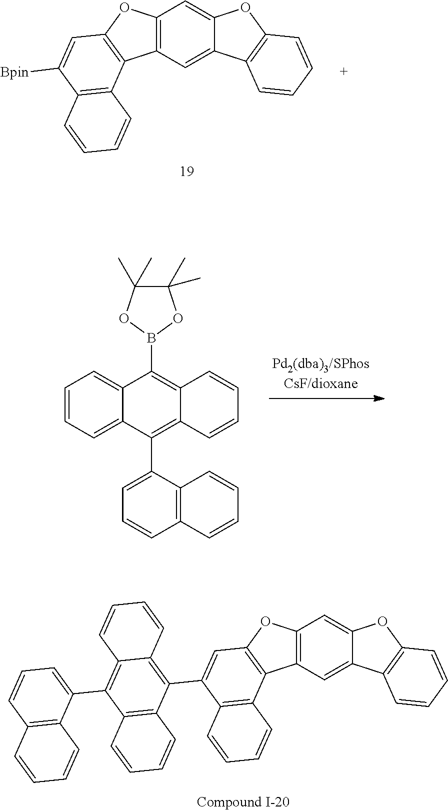

5-(10-(1-Naphthyl)-anthracene-9-yl)benzofuro[3,2-f]naphtho[2,1-b]benzofuran (Compound I-20).

A mixture of 9-bromo-10-phenylanthracene (10.0 g, 26.09 mmole), 5-(4,4,5,5-tetramethyl-1,3,2-dioxaborolan-2-yl)benzo[d]naphtho[1,2-d′]benzo[1,2-b:5,4-b′]difuran 19 (11.22 g, 26.09 mmole), Pd2(dba)3 (478 mg, 0.5218 mmole), SPhos (428 mg, 1.044 mmole), potassium phosphate (16.61 g, 78.27 mmole), toluene (200 ml), water (40 ml), ethanol (80 ml) was heated at reflux with stirring under nitrogen atmosphere for 16 hours. Reaction mixture cooled down, precipitate filtered, washed with toluene, water, methanol, dried in vacuum to give crude product. The product was dissolved in hot chloroform (800 ml), passed through a filter filled with silica gel, florisil and basic alumina eluating with hot chloroform. Chloroform evaporated to volume 100 ml, precipitates collected by filtration portion-wise (13.96 g), stirred in a mixture of chloroform and methanol (1:2) for 30 min., filtered, dried in vacuum to give Compound I-2 with purity greater than 99.74% by UPLC. Product with purity greater than 99.99% was obtained by stirring solid product in a mixture of hot toluene and 1,2-dichlorobenzene, filtration, washing with toluene, hexanes. MS: MH+612. ¹H-NMR (CD₂Cl₂, 500 MHz): 7.22-7.59 (m, 14H), 7.68-7.74 (m, 2H), 7.79-7.83 (m, 1H), 7.85-7.89 (m, 1H), 7.996 and 8.03 (s, 1H, atropoisomers), 8.02 and 8.07 (s, 1H, atropoisomers), 8.09-8.12 (m, 1H), 8.17 (d, 2H, J=8 Hz), 8.29 (d, 2H, J=8 Hz), 9.02-9.04 (m, 1H), 9.13 (s, 1H).

### Synthesis Example 5

This example illustrates the preparation of a compound having Formula I, Compound I-21

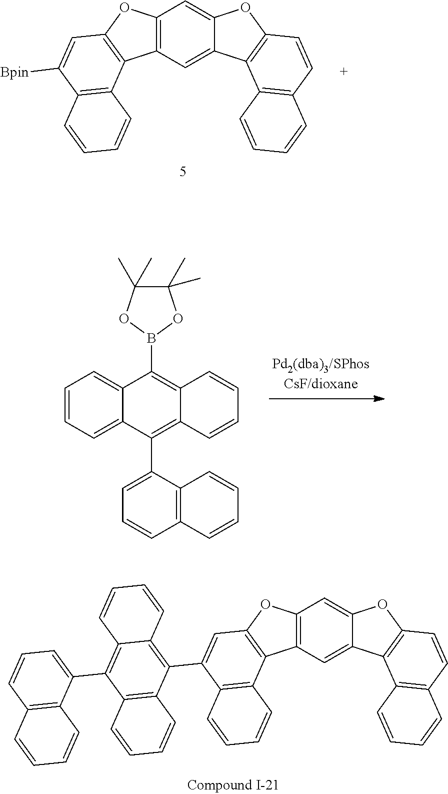

5-(10-(1-Naphthyl)-anthracene-9-yl)-dinaphtho[1,2-d:1′,2′-d′]benzo[1,2-b:5,4-b′]difuran (Compound I-21).

A mixture of 9-bromo-10-phenylanthracene (2.03 g, 5.28 mmole), 5-(4,4,5,5-tetramethyl-1,3,2-dioxaborolan-2-yl)-dinaphtho[1,2-d:1′,2′-d′]benzo[1,2-b:5,4-b′]difuran 5 (2.56 g, 5.28 mmole), Pd2(dba)3 (97 mg, 0.106 mmole), SPhos (325 mg, 0.79 mmole), potassium phosphate (3.14 g, 14.8 mmole), toluene (125 ml), water (25 ml), ethanol (50 ml) was heated at reflux with stirring under nitrogen atmosphere for overnight. Reaction mixture cooled down, precipitate filtered, washed with toluene, hexanes, water, dried to give 3.16 g of crude product. The product was dissolved in hot 1,2-dichlorobenzene (40 ml), passed through a filter filled with basic alumina, florisil, silica gel washing with 1,2-dichlorobenzene. Precipitate collected by filtration, washed with small amount of 1,2-dichlorobenzene, treated with a mixture of dichloromethane—methanol (1:1) to give 1.98 g of product with purity 99.15% by UPLC. The product was further recrystallized from 40 ml of hot 1,2-dichlorobenzene, precipitate collected by filtration, washed with 1,2-dichlorobenzene, hexanes, dried in vacuum to give 1.78 g of Compound I-21 with purity 99.98% by UPLC. MS: MH+662. ¹H-NMR (CDCl₃, 500 MHz): 7.19-7.53 (m, 12H), 7.65-7.72 (m, 2H), 7.75-7.79 (m, 1H), 7.88 (d, 1H, J=9 Hz), 7.89-7.97 (m, 3H), 8.01-8.08 (m, 4H), 8.13 (t, 2H, J=7 Hz), 8.98 (d, 1H, J=8 Hz), 9.06-9.09 (m, 1H), 9.46 (s, 1H).

### Synthesis Example 6

This example illustrates the preparation of a compound having Formula I, Compound I-22.

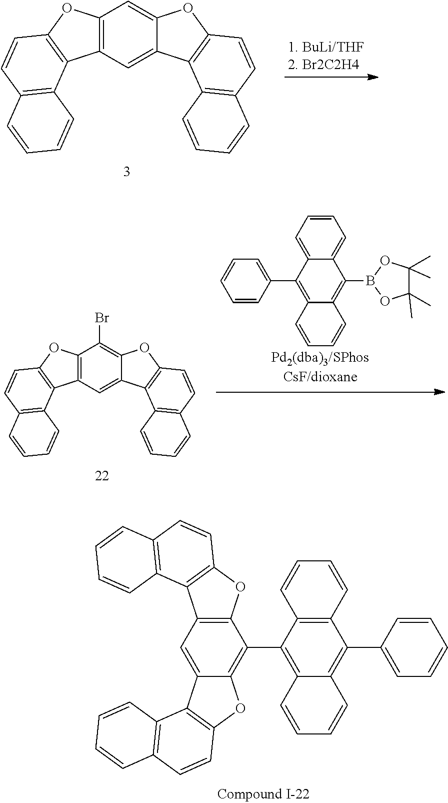 [SMILES: c1ccc2c(c1)ccc1oc3cc4oc5ccc6ccccc6c5c4cc3c12 ; Brc1c2oc3ccc4ccccc4c3c2cc2c1oc1ccc3ccccc3c12 ; CC1(C)OB(c2c3ccccc3c(-c3ccccc3)c3ccccc23)OC1(C)C ; c1ccc(-c2c3ccccc3c(-c3c4oc5ccc6ccccc6c5c4cc4c3oc3ccc5ccccc5c34)c3ccccc23)cc1]

(a) 8-Bromo-dinaphtho[1,2-d:1′,2′-d′]benzo[1,2-b:5,4-b′]difuran (22).

Dinaphtho[1,2-d:1′,2′-d′]benzo[1,2-b:5,4-b′]difuran 3 (3.24 g, 9.04 mmole) was dissolved in anhydrous tetrahydrofuran (648 ml) under nitrogen atmosphere, cooled down to ambient temperature and to resulting solution was added n-butyllithium (6.75 ml of 1.6 M solution in hexanes, 1.2 equivalents). The mixture was kept at ambient temperature for overnight followed by addition of 1,2-dibromoethane (5.09 g, 27.1 mmole). Resulting precipitate collected by filtration after 30 min, washed with tetrahydrofuran, water, methanol, dried to give 2.07 g of crude product that was further purified by crystallization from hot 1,2-dichlorobenzene (41 ml), collecting precipitate, washing with 1,2-dichlorobenzene, hexanes to give 1.83 g of compound 22 with purity 99.64% by UPLC. MS: MH+=439. UV-vis (acetonitrile-water): 359, 342, 261 nm.

(b) 8-(10-phenyl-9-anthracenyl)-dinaphtho[1,2-d:1′,2′-d′]benzo[1,2-b:5,4-b′]difuran (Compound I-22).

A mixture of 4,4,5,5-tetramethyl-2-(10-phenyl-9-anthracenyl)-1,3,2-dioxaborolane (1.49 g, 3.91 mmole), 8-bromo-dinaphtho[1,2-d:1′,2′-d′]benzo[1,2-b:5,4-b′]difuran 22 (1.71 g, 3.91 mmole), Pd₂(dba)₃ (72 mg, 0.078 mmole), SPhos (128 mg, 0.313 mmole), cesium fluoride (2.98 g, 19.6 mmole), toluene (110 ml), ethanol (44 ml), water (22 ml) was heated at reflux with stirring under nitrogen atmosphere for 3 days. Reaction mixture filtered hot, precipitate washed with toluene, hexanes, water, methanol, dried in vacuum to give 1.60 g of crude product. The product was dissolved in hot 1,2-dichlorobenzene (40 ml), filtered through a filter filled with basic alumina, florisil, silica gel eluating with 1,2-dichlorobenzene. Precipitate collected by filtration, washed with 1,2-dichlorobenzene, hexanes, dried to give 1.14 g of the product that was further recrystallized from hot 1,2-dichlorobenzene (100 ml), precipitate collected by filtration, washed with 1,2-dichlorobenzene, hexanes to give Compound I-22 (0.67 g). MS: MH+612.

### Device Examples

### (1) Materials

ET-1 is a triazine derivative
ET-2 is a fluorene substituted triazine
LiQ is lithium quinolate.
HAT-CN is 1,4,5,8,9,12-hexaazatriphenylenehexacarbonitrile.
Dopant-1 is a di-arylamino pyrene
Dopant-2, shown below, can be made as described in J. Mater. Chem. C, 2019, 7, 3082

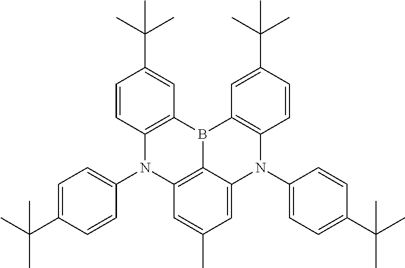 [SMILES: Cc1cc2c3c(c1)N(c1ccc(C(C)(C)C)cc1)c1ccc(C(C)(C)C)cc1B3c1cc(C(C)(C)C)ccc1N2c1ccc(C(C)(C)C)cc1]

Host A, shown below, can be made as described in U.S. Pat. No. 8,084,146

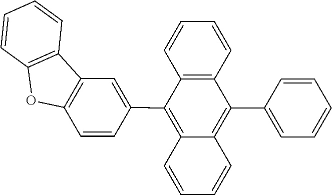 [SMILES: c1ccc(-c2c3ccccc3c(-c3ccc4oc5ccccc5c4c3)c3ccccc23)cc1]

HTM-1 is a fluorene substituted arylamine
HTM-2 is a mono-arylamino phenanthrene.
HTM-3 is an aryl carbazole derivative
HTM-4 is a dibenzofuranyl arylamine
HTM-5 is a carbazole-substituted triarylamine
HTM-6 is a carbazole-substituted aryldiamine

### (2) Devices

The emissive layers were deposited by vapor deposition as detailed below. In all cases, prior to use the substrates were cleaned ultrasonically in detergent, rinsed with water and subsequently dried in nitrogen.

### (3) Device Characterization

The OLED devices were characterized by measuring their (1) current-voltage (I-V) curves, (2) electroluminescence radiance versus voltage, and (3) electroluminescence spectra versus voltage. All three measurements were performed at the same time and controlled by a computer. The current efficiency of the device at a certain voltage is determined by dividing the electroluminescence radiance of the LED by the current density needed to run the device. The unit is a cd/A. The power efficiency is the current efficiency divided by the operating voltage. The unit is Im/W.

### Device Examples 1-4

These examples illustrate the use of a compound having Formula I as the host material in the photoactive layer of a device. The devices were bottom-emission devices made by thermal evaporation.

Bottom-emission devices were fabricated on patterned indium tin oxide (ITO) coated glass substrates. Cleaned substrates were loaded into a vacuum chamber. Once pressure reached 5×10⁻⁷ Torr or below, they received thermal evaporations of the hole injection material, a first hole transport material, a second hole transport material, the photoactive and host materials, electron transport materials and electron injection material sequentially. The bottom-emission devices were thermally evaporated with Al cathode material. The chamber was then vented, and the devices were encapsulated using a glass lid, desiccant, and UV curable epoxy.

The device had the structure, in order (unless otherwise specified, all ratios are by weight and all percentages are by weight, based on the total weight of the layer):
Glass substrate

Anode: ITO (50 nm)
HIL: HAT-CN (10 nm)
HTL1: HTM-1, with the thickness shown in the table below
HTL2: shown in the table below
EML: host Compound I-1, in a 20:1 weight ratio with Dopant-1 (25 nm)
ETL1: ET-1 (5 nm)
ETL2: ET-2:LiQ 1:1 (22 nm)
EIL:LiQ (3 nm)
Cathode: Al (100 nm)

TABLE 1
Device results

| | HTL1 | HTL2 | | | | | |
|---|---|---|---|---|---|---|---|
| Dev. | Thick- | | Thick- | | | | |
| Ex. | ness | Material | ness | V | CE | CIEx | CIEy |
| 1 | 170 | HTM-2 | 20 | 3.9 | 6.1 | 0.140 | 0.10 |
| 2 | 180 | HTM-3 | 10 | 3.9 | 6.9 | 0.137 | 0.11 |
| 3 | 180 | HTM-4 | 10 | 3.9 | 5.8 | 0.136 | 0.12 |
| 4 | 170 | HTM-4 | 20 | 4.0 | 5.7 | 0.136 | 0.12 |

Thickness is the layer thickness in nm; V is the voltage at 10 mA/cm²; All other data at 1000 nits. CE is the current efficiency in cd/A; CIEx and CIEy are the x and y color coordinates according to the C.I.E. chromaticity scale (Commission Internationale de L'Eclairage, 1931).

### Device Examples 5-8 and Comparative Example A

Bottom-emission devices were fabricated as described above for Device Examples 1-4.

The device had the structure, in order (unless otherwise specified, all ratios are by weight and all percentages are by weight, based on the total weight of the layer):
Glass substrate
Anode: ITO (50 nm)
HIL: HAT-CN (10 nm)\HTM-6 (90 nm)\HAT-CN (5 nm)
HTL1: HTM-1 (71 nm)
HTL2: HTM-5 (10 nm)
EML: host as shown in Table 2, in a 32:1 ratio with Dopant-2 (25 nm)
ETL1: ET-2:LiQ 1:1 (27 nm)
EIL: LiQ (3 nm)
Cathode: Al (100 nm)

TABLE 2
Device results

| Dev. Ex. | Host | V10 | CE | CIEx | CIEy |
|---|---|---|---|---|---|
| Comp. A | Host A | 5.0 | 5.8 | 0.132 | 0.090 |
| 5 | Compound | 3.6 | 6.8 | 0.134 | 0.091 |
| | I-2 | | | | |
| 6 | Compound | 3.8 | 7.4 | 0.135 | 0.092 |
| | I-20 | | | | |
| 7 | Compound | 3.5 | 6.7 | 0.134 | 0.094 |
| | I-21 | | | | |
| 8 | Compound | 4.5 | 7.6 | 0.134 | 0.094 |
| | I-22 | | | | |

V10 is the driving voltage at 10 mA/cm²; All other data at 1000 nits. CIEx and CIEy are the x and y color coordinates according to the C.I.E. chromaticity scale (Commission Internationale de L'Eclairage, 1931); CE is the current efficiency in cd/A.

It can be seen from Table 2, that devices with the compounds of the present invention as host have lower driving voltage and higher current efficiency compared to the device with comparative Host A.

Note that not all of the activities described above in the general description or the examples are required, that a portion of a specific activity may not be required, and that one or more further activities may be performed in addition to those described. Still further, the order in which activities are listed are not necessarily the order in which they are performed.

In the foregoing specification, the concepts have been described with reference to specific embodiments. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of invention.

Benefits, other advantages, and solutions to problems have been described above with regard to specific embodiments. However, the benefits, advantages, solutions to problems, and any feature(s) that may cause any benefit, advantage, or solution to occur or become more pronounced are not to be construed as a critical, required, or essential feature of any or all the claims.

It is to be appreciated that certain features are, for clarity, described herein in the context of separate embodiments, may also be provided in combination in a single embodiment. Conversely, various features that are, for brevity, described in the context of a single embodiment, may also be provided separately or in any subcombination. Further, reference to values stated in ranges include each and every value within that range.

What is claimed is:

1. A compound having Formula I Formula I 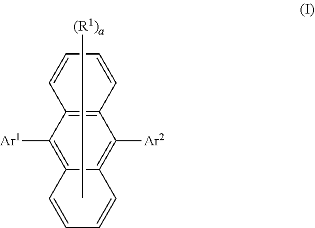
wherein:
Ar¹ is selected from the group consisting of hydrocarbon aryl groups, heteroaryl groups, and substituted derivatives thereof;
Ar² is selected from the group consisting of Formula IA, Formula IB, Formula IC, Formula IAa, Formula IBb, and Formula ICc 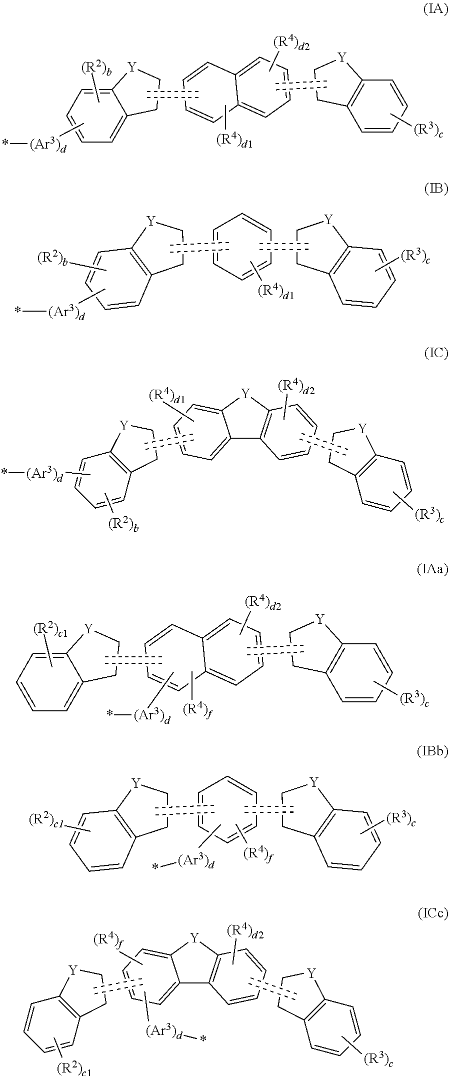
wherein:
Ar³ is the same or different at each occurrence and is selected from the group consisting of phenyl, naphthyl, and substituted derivatives thereof;
Y is the same or different at each occurrence and is selected from the group consisting of CR^{a}R^{b}, O, S, and Se, with the proviso that at least one Y is selected from the group consisting of O, S, and Se;
R^{a} and R^{b} are the same or different at each occurrence and are selected from the group consisting of alkyl, silyl, germyl, hydrocarbon aryl, heteroaryl, and substituted derivatives thereof, where R^{a} and R^{b} can be joined to form a cyclic group selected from the group consisting of cycloalkyl, silacycloalkyl, spirofluorenyl, silaspirofluorenyl, or a substituted derivative thereof;
R¹-R⁴ are the same or different at each occurrence and are selected from the group consisting of D, F, CN, alkyl, fluoroalkyl, hydrocarbon aryl, heteroaryl, silyl, germyl, deuterated alkyl, deuterated partially-fluorinated alkyl, deuterated hydrocarbon aryl, deuterated heteroaryl, deuterated heteroaryl deuterated silyl, and deuterated germyl, where adjacent R² and/or R³ groups can be joined together to form a fused aromatic ring;
a is an integer from 0-8;
b is an integer from 0-3;
c is an integer from 0-4;
d, d1, and d2 are the same or different and are an integer from 0-2;
f is an integer from 0-1;
a double dashed line between two rings indicates that the rings are fused together in any orientation; and
* indicates a point of attachment in the identified formula;
with the proviso that:
in Formula IB and Formula IC there is at least one naphthyl group formed by R² or R³ groups, where the naphthyl group may have one or more substituents selected from the group consisting of D, F, CN, alkyl, fluoroalkyl, hydrocarbon aryl, heteroaryl, silyl, germyl, deuterated alkyl, deuterated partially-fluorinated alkyl, deuterated hydrocarbon aryl, deuterated heteroaryl, deuterated heteroaryl deuterated silyl, and deuterated germyl.

2. The compound according to claim 1, wherein at least one Y=O

3. The compound according to claim 1, wherein all Y=O.

4. The compound according to claim 1, wherein Ar² is selected from the group consisting of a hydrocarbon aryl having 6-30 ring carbons and a deuterated analog thereof.

5. The compound according to claim 1, wherein Ar² is selected from the group consisting of Formula IA has Formula IA-1, Formula IA-2, Formula IA-3, and Formula IA-4 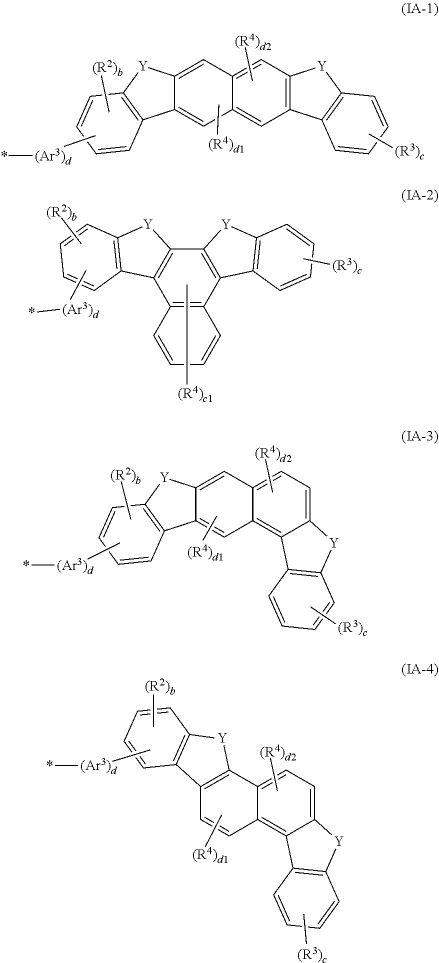
where:
c1 is an integer from 0-4.

6. The compound according to claim 1, wherein Ar² is selected from the group consisting of Formula IB has Formula IB-1, Formula IB-2, and Formula IB-3 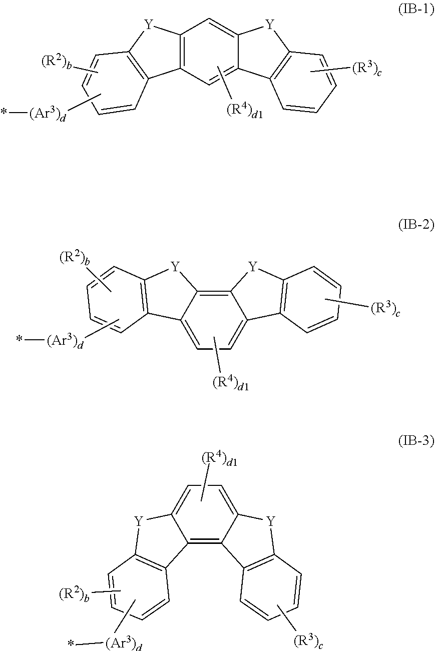
where:
c1 is an integer from 0-4;
with the proviso that there is at least one naphthyl group formed by R² or R³ groups, where the naphthyl group may have one or more substituents selected from the group consisting of D, F, CN, alkyl, fluoroalkyl, hydrocarbon aryl, heteroaryl, silyl, germyl, deuterated alkyl, deuterated partially-fluorinated alkyl, deuterated hydrocarbon aryl, deuterated heteroaryl, deuterated heteroaryl deuterated silyl, and deuterated germyl.

7. The compound according to claim 1, wherein Ar² is selected from the group consisting of Formula IC-1, Formula IC-2, Formula IC-3, Formula IC-4, Formula IC-5, Formula IC-6, Formula IC-7, Formula IC-8, and Formula IC-9 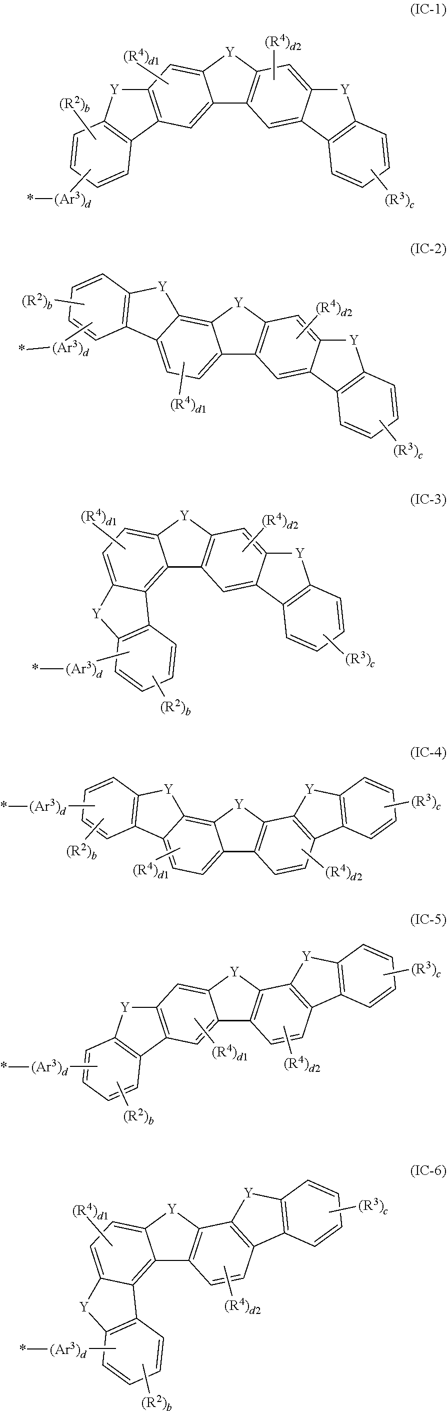 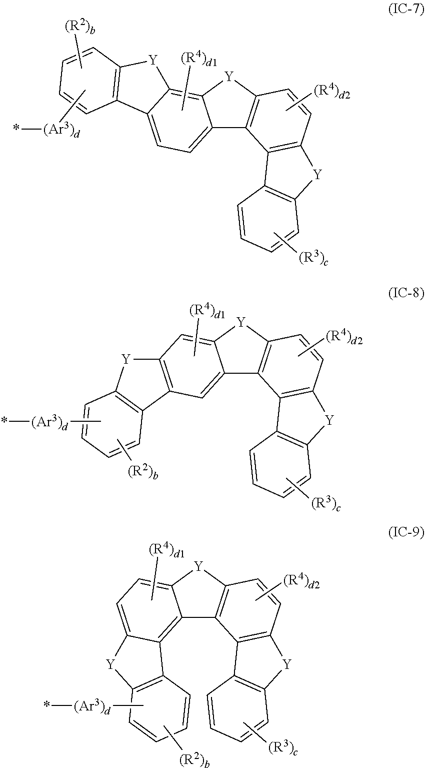
where:
c1 is an integer from 0-4;
with the proviso that there is at least one naphthyl group formed by R² or R³ groups, where the naphthyl group may have one or more substituents selected from the group consisting of D, F, CN, alkyl, fluoroalkyl, hydrocarbon aryl, heteroaryl, silyl, germyl, deuterated alkyl, deuterated partially-fluorinated alkyl, deuterated hydrocarbon aryl, deuterated heteroaryl, deuterated heteroaryl deuterated silyl, and deuterated germyl.

8. An organic electronic device comprising a first electrical contact, a second electrical contact and a photoactive layer therebetween, wherein the photoactive layer comprises a compound according to claim 1.
